(19) United States
(12) Patent Application Publication
(10) Pub. No.: US 2008/0009534 A1
(43) Pub. Date: Jan. 10, 2008

(54) SUBSTITUTED ACID DERIVATIVES USEFUL AS ANTIDIABETIC AND ANTIOBESITY AGENTS AND METHOD

(75) Inventors: Peter T.W. Cheng, Princeton, NJ (US); Sean Chen, Princeton, NJ (US); Xinhua Qian, Flemington, NJ (US); Rajendra P. Deshpande, Hillsborough, NJ (US); Erqing Tang, Somerset, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/773,564

(22) Filed: Jul. 5, 2007

Related U.S. Application Data
(60) Provisional application No. 60/819,110, filed on Jul. 7, 2006.

(51) Int. Cl. A61K 31/42; A61P 43/00; C07D 263/30

(52) U.S. Cl. 514/374; 548/235

(57) ABSTRACT

Compounds are provided which have the structure of Formula (I): 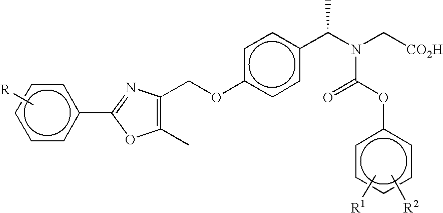
wherein R is hydrogen or C₁-C₄ alkyl; and each of R¹ and R² is independently hydrogen, C₁-C₄ alkyl, halo or C₁-C₄ alkoxy, and salts thereof, which compounds are useful as antidiabetic, hypolipidemic, and antiobesity agents.

### CROSS-REFERENCE TO RELATED APPLICATION

This application claims priority benefit under Title 35 § 119(e) of U.S. provisional Application No. 60/819,1 10, filed Jul. 7, 2006, the contents of which are herein incorporated by reference in its entirety.

### FIELD OF THE INVENTION

The present invention relates to novel substituted acid derivatives which modulate blood glucose levels, triglyceride levels, insulin levels and non-esterified fatty acid (NEFA) levels, and thus are particularly useful in the treatment of diabetes and obesity, and to a method for treating diabetes, especially Type 2 diabetes, as well as hyperglycemia, hyperinsulinemia, dyslipidemia, obesity, atherosclerosis and related diseases employing such substituted acid derivatives alone or in combination with another antidiabetic agent and/or an anti-dyslipidemic agent.

### DESCRIPTION OF THE INVENTION

In accordance with the present invention, substituted acid derivatives are provided which have the Formula (I): 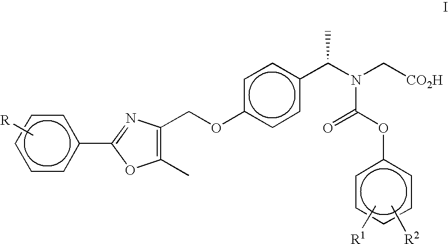
wherein R is hydrogen or C₁-C₄ alkyl; and each of R¹ and R² is independently hydrogen, C₁-C₄ alkyl, halo or C₁-C₄ alkoxy, and salts thereof.

A preferred compound of the present invention has the structure of Formula (Ia):

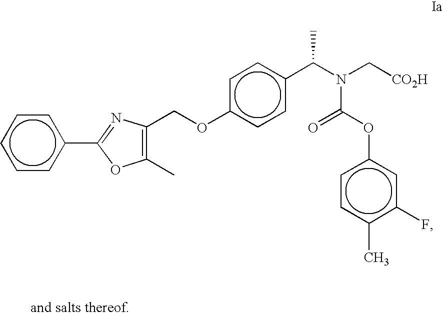

Another preferred compound of the instant invention has the structure of Formula (Ib):

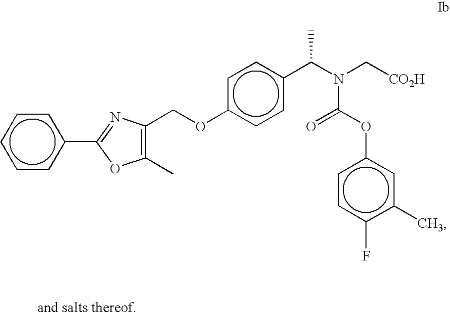

Yet another preferred compound of the instant invention has the structure of Formula (Ic):

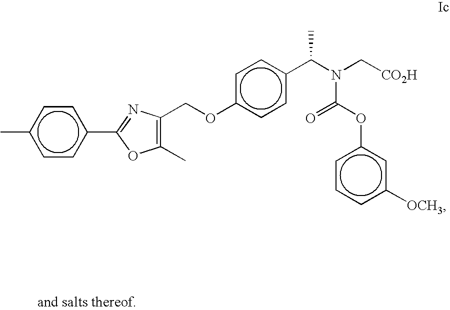

In addition, in accordance with the present invention, a method is provided for treating diabetes, especially Type 2 diabetes, and related diseases such as insulin resistance, hyperglycemia, hyperinsulinemia, elevated blood levels of fatty acids or glycerol, dyslipidemia, obesity, hypertriglyceridemia, inflammation, Syndrome X, diabetic complications, dysmetabolic syndrome, atherosclerosis, and related diseases wherein a therapeutically effective amount of a compound of Formula I is administered to a human patient in need of treatment.

For ease of reference, when Formula I is mentioned in the description of the invention, it is intended that Formulae Ia, Ib and Ic are also included in the scope thereof.

In addition, in accordance with the present invention, a method is provided for treating early malignant lesions (such as ductal carcinoma in situ of the breast and lobular carcinoma in situ of the breast), premalignant lesions (such as fibroadenoma of the breast and prostatic intraepithelial neoplasia (PIN), liposarcomas and various other epithelial tumors (including breast, prostate, colon, ovarian, gastric and lung), irritable bowel syndrome, Crohn's disease, gastric ulceritis, and osteoporosis and proliferative diseases such as psoriasis, wherein a therapeutically effective amount of a compound of Formula I is administered to a human patient in need of treatment.

Furthermore, in accordance with the present invention, a method is provided for treating diabetes and related diseases as defined above and hereinafter, wherein a therapeutically effective amount of a combination of a compound of Formula I and another type anti-diabetic agent and/or a hypolipidemic agent, and/or lipid modulating agent and/or other type of therapeutic agent, is administered to a human patient in need of treatment.

In the above methods of the invention, the compound of Formula I will be employed in a weight ratio to the anti-diabetic agent (depending upon its mode of operation) within the range from about 0.01:1 to about 100:1, preferably from about 0.5:1 to about 10:1.

The conditions, diseases, and maladies collectively referenced to as “Syndrome X” or Dysmetabolic Syndrome are detailed in Johannsson, J. Clin. Endocrinol. Metab., 82:727-734 (1997) and other publications.

The term “diabetes and related diseases” refers to Type II diabetes, Type I diabetes, impaired glucose tolerance, obesity, hyperglycemia, Syndrome X, dysmetabolic syndrome, diabetic complications and hyperinsulinemia.

The conditions, diseases and maladies collectively referred to as “diabetic complications” include retinopathy, neuropathy and nephropathy, and other known complications of diabetes.

The term “other type(s) of therapeutic agents” as employed herein refers to one or more anti-diabetic agents (other than compounds of Formula I), one or more anti-obesity agents, and/or one or more lipid-lowering agents, one or more lipid modulating agents (including anti-atherosclerosis agents), and/or one or more anti-platelet agents, one or more agents for treating hypertension, one or more anti-cancer drugs, one or more agents for treating arthritis, one or more anti-osteoporosis agents, one or more anti-obesity agents, one or more agents for treating immunomodulatory diseases, and/or one or more agents for treating anorexia nervosa.

The term “lipid-modulating” agent as employed herein refers to agents which lower LDL and/or raise HDL and/or lower triglycerides and/or lower total cholesterol and/or other known mechanisms for therapeutically treating lipid disorders.

Unless otherwise indicated, the term “lower alkyl”, “alkyl” or “alk” as employed herein alone or as part of another group includes both straight and branched chain hydrocarbons, containing 1 to 20 carbons, preferably 1 to 10 carbons, more preferably 1 to 8 carbons, in the normal chain, and may optionally include an oxygen or nitrogen in the normal chain, such as methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, the various branched chain isomers thereof, and the like as well as such groups including 1 to 4 substituents such as halo, for example F, Br, Cl or I or CF₃, alkoxy, aryl, aryloxy, aryl(aryl) or diaryl, arylalkyl, arylalkyloxy, alkenyl, cycloalkyl, cycloalkylalkyl, cycloalkylalkyloxy, amino, hydroxy, hydroxyalkyl, acyl, heteroaryl, heteroaryloxy, cycloheteroalkyl, arylheteroaryl, arylalkoxycarbonyl, heteroarylalkyl, heteroarylalkoxy, aryloxyalkyl, aryloxyaryl, alkylamido, alkanoylamino, arylcarbonylamino, nitro, cyano, thiol, haloalkyl, trihaloalkyl and/or alkylthio and/or any of the R³ groups.

Unless otherwise indicated, the term “cycloalkyl” as employed herein alone or as part of another group includes saturated or partially unsaturated (containing 1 or 2 double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 10 carbons, forming the ring and which may be fused to 1 or 2 aromatic rings as described for aryl, which include cyclopropyl, cyclobutyl, cyclopentyl, cyclohexyl, cycloheptyl, cyclooctyl, cyclodecyl and cyclododecyl, cyclohexenyl, 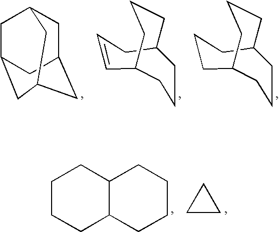
any of which groups may be optionally substituted with 1 to 4 substituents such as halogen, alkyl, alkoxy, hydroxy, aryl, aryloxy, arylalkyl, cycloalkyl, alkylamido, alkanoylamino, oxo, acyl, arylcarbonylamino, amino, nitro, cyano, thiol and/or alkylthio and/or any of the substituents for alkyl.

The term “cycloalkenyl” as employed herein alone or as part of another group refers to cyclic hydrocarbons containing 3 to 12 carbons, preferably 5 to 10 carbons and 1 or 2 double bonds. Exemplary cycloalkenyl groups include cyclopentenyl, cyclohexenyl, cycloheptenyl, cyclooctenyl, cyclohexadienyl, and cycloheptadienyl, which may be optionally substituted as defined for cycloalkyl.

The term “cycloalkylene” as employed herein refers to a “cycloalkyl” group which includes free bonds and thus is a linking group such as 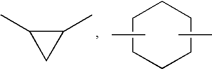
and the like, and may optionally be substituted as defined above for “cycloalkyl”.

The term “alkanoyl” as used herein alone or as part of another group refers to alkyl linked to a carbonyl group.

Unless otherwise indicated, the term “lower alkenyl” or “alkenyl” as used herein by itself or as part of another group refers to straight or branched chain radicals of 2 to 20 carbons, preferably 2 to 12 carbons, and more preferably 1 to 8 carbons in the normal chain, which include one to six double bonds in the normal chain, and may optionally include an oxygen or nitrogen in the normal chain, such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, 4,8,12-tetradecatrienyl, and the like, and which may be optionally substituted with 1 to 4 substituents, namely, halogen, haloalkyl, alkyl, alkoxy, alkenyl, alkynyl, aryl, arylalkyl, cycloalkyl, amino, hydroxy, heteroaryl, cycloheteroalkyl, alkanoylamino, alkylamido, arylcarbonylamino, nitro, cyano, thiol, alkylthio and/or any of the substituents for alkyl set out herein.

Unless otherwise indicated, the term “lower alkynyl” or “alkynyl” as used herein by itself or as part of another group refers to straight or branched chain radicals of 2 to 20 carbons, preferably 2 to 12 carbons and more preferably 2 to 8 carbons in the normal chain, which include one triple bond in the normal chain, and may optionally include an oxygen or nitrogen in the normal chain, such as 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, 3-nonynyl, 4-decynyl,3-undecynyl, 4-dodecynyl and the like, and which may be optionally substituted with 1 to 4 substituents, namely, halogen, haloalkyl, alkyl, alkoxy, alkenyl, alkynyl, aryl, arylalkyl, cycloalkyl, amino, heteroaryl, cycloheteroalkyl, hydroxy, alkanoylamino, alkylamido, arylcarbonylamino, nitro, cyano, thiol, and/or alkylthio, and/or any of the substituents for alkyl set out herein.

The terms “arylalkenyl” and “arylalkynyl” as used alone or as part of another group refer to alkenyl and alkynyl groups as described above having an aryl substituent.

Where alkyl groups as defined above have single bonds for attachment to other groups at two different carbon atoms, they are termed “alkylene” groups and may optionally be substituted as defined above for “alkyl”.

Where alkenyl groups as defined above and alkynyl groups as defined above, respectively, have single bonds for attachment at two different carbon atoms, they are termed “alkenylene groups” and “alkynylene groups”, respectively, and may optionally be substituted as defined above for “alkenyl” and “alkynyl”.

(CH₂)ₓ, (CH₂)ₘ, (CH₂)ₙ or (CH₂)_{y} includes alkylene, allenyl, alkenylene or alkynylene groups, as defined herein, each of which may optionally include an oxygen or nitrogen in the normal chain, which may optionally include 1, 2, or 3 substituents which include alkyl, alkenyl, halogen, cyano, hydroxy, alkoxy, amino, thioalkyl, keto, C₃-C₆ cycloalkyl, alkylcarbonylamino or alkylcarbonyloxy; the alkyl substituent may be an alkylene moiety of 1 to 4 carbons which may be attached to one or two carbons in the (CH₂)ₓ or (CH₂)ₘ or (CH₂)ₙ group to form a cycloalkyl group therewith.

Examples of (CH₂)ₓ, (CH₂)ₘ, (CH₂)ₙ, (CH₂)_{y}, alkylene, alkenylene and alkynylene include

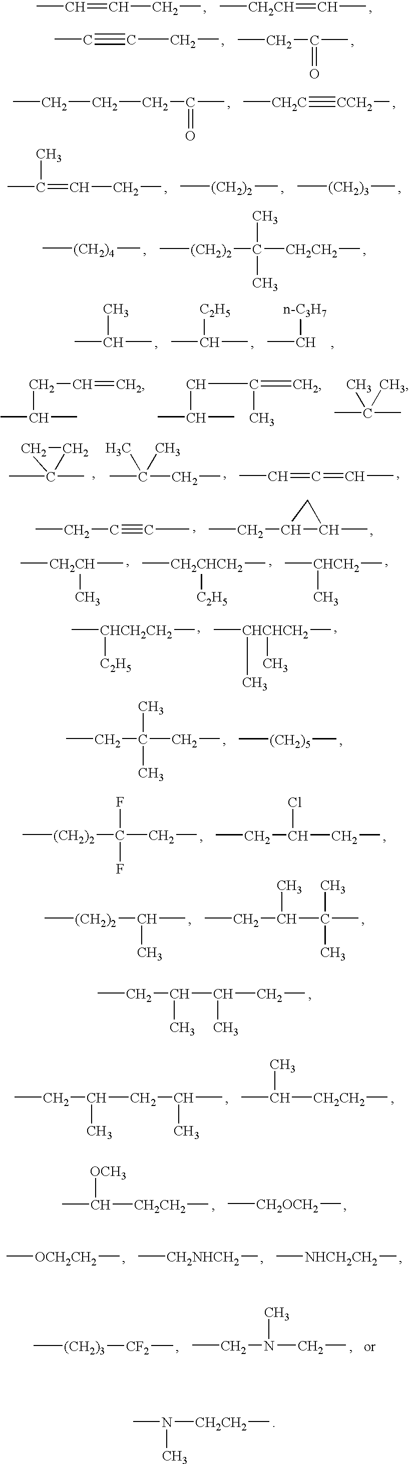

The term “halogen” or “halo” as used herein alone or as part of another group refers to chlorine, bromine, fluorine, and iodine as well as CF₃, with chlorine or fluorine being preferred.

The term “metal ion” refers to alkali metal ions such as sodium, potassium or lithium and alkaline earth metal ions such as magnesium and calcium, as well as zinc and aluminum.

Unless otherwise indicated, the term “aryl” or the group 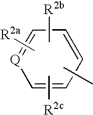
where Q is C, as employed herein alone or as part of another group refers to monocyclic and bicyclic aromatic groups containing 6 to 10 carbons in the ring portion (such as phenyl or naphthyl including 1-naphthyl and 2-naphthyl) and may optionally include one to three additional rings fused to a carbocyclic ring or a heterocyclic ring (such as aryl, cycloalkyl, heteroaryl or cycloheteroalkyl rings
for example 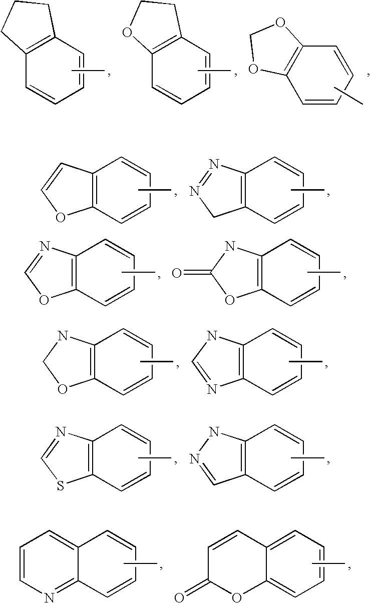
and may be optionally substituted through available carbon atoms with 1, 2, or 3 groups selected from hydrogen, halo, haloalkyl, alkyl, haloalkyl, alkoxy, haloalkoxy, alkenyl, trifluoromethyl, trifluoromethoxy, alkynyl, cycloalkyl-alkyl, cycloheteroalkyl, cycloheteroalkylalkyl, aryl, heteroaryl, arylalkyl, aryloxy, aryloxyalkyl, arylalkoxy, alkoxycarbonyl, arylcarbonyl, arylalkenyl, aminocarbonylaryl, arylthio, arylsulfinyl, arylazo, heteroarylalkyl, heteroarylalkenyl, heteroarylheteroaryl, heteroaryloxy, hydroxy, nitro, cyano, amino, substituted amino wherein the amino includes 1 or 2 substituents (which are alkyl, aryl or any of the other aryl compounds mentioned in the definitions), thiol, alkylthio, arylthio, heteroarylthio, arylthioalkyl, alkoxyarylthio, alkylcarbonyl, arylcarbonyl, alkylaminocarbonyl, arylaminocarbonyl, alkoxycarbonyl, aminocarbonyl, alkylcarbonyloxy, arylcarbonyloxy, alkylcarbonylamino, arylcarbonylamino, arylsulfinyl, arylsulfinylalkyl, arylsulfonylamino or arylsulfonaminocarbonyl and/or any of the substituents for alkyl set out herein.

Unless otherwise indicated, the term “lower alkoxy”, “alkoxy”, “aryloxy” or “aralkoxy” as employed herein alone or as part of another group includes any of the above alkyl, aralkyl or aryl groups linked to an oxygen atom.

Unless otherwise indicated, the term “substituted amino” as employed herein alone or as part of another group refers to amino substituted with one or two substituents, which may be the same or different, such as alkyl, aryl, arylalkyl, heteroaryl, heteroarylalkyl, cycloheteroalkyl, cycloheteroalkylalkyl, cycloalkyl, cycloalkylalkyl, haloalkyl, hydroxyalkyl, alkoxyalkyl or thioalkyl. These substituents may be further substituted with a carboxylic acid and/or any of the substituents for alkyl as set out above. In addition, the amino substituents may be taken together with the nitrogen atom to which they are attached to form 1-pyrrolidinyl, 1-piperidinyl, 1-azepinyl, 4-morpholinyl, 4-thiamorpholinyl, 1-piperazinyl, 4-alkyl-1-piperazinyl, 4-arylalkyl-1-piperazinyl, 4-diarylalkyl-1-piperazinyl, 1-pyrrolidinyl, 1-piperidinyl, or 1-azepinyl, optionally substituted with alkyl, alkoxy, alkylthio, halo, trifluoromethyl or hydroxy.

Unless otherwise indicated, the term “lower alkylthio”, alkylthio”, “arylthio” or “aralkylthio” as employed herein alone or as part of another group includes any of the above alkyl, aralkyl or aryl groups linked to a sulfur atom.

Unless otherwise indicated, the term “lower alkylamino”, “alkylamino”, “arylamino”, or “arylalkylamino” as employed herein alone or as part of another group includes any of the above alkyl, aryl or arylalkyl groups linked to a nitrogen atom.

Unless otherwise indicated, the term “acyl” as employed herein by itself or part of another group, as defined herein, refers to an organic radical linked to a carbonyl 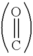
group; examples of acyl groups include any of the R³ groups attached to a carbonyl, such as alkanoyl, alkenoyl, aroyl, aralkanoyl, heteroaroyl, cycloalkanoyl, cycloheteroalkanoyl and the like.

Unless otherwise indicated, the term “cycloheteroalkyl” as used herein alone or as part of another group refers to a 5-, 6- or 7-membered saturated or partially unsaturated ring which includes 1 to 2 hetero atoms such as nitrogen, oxygen and/or sulfur, linked through a carbon atom or a heteroatom, where possible, optionally via the linker (CH₂)ₚ (where p is 1, 2 or 3), such as 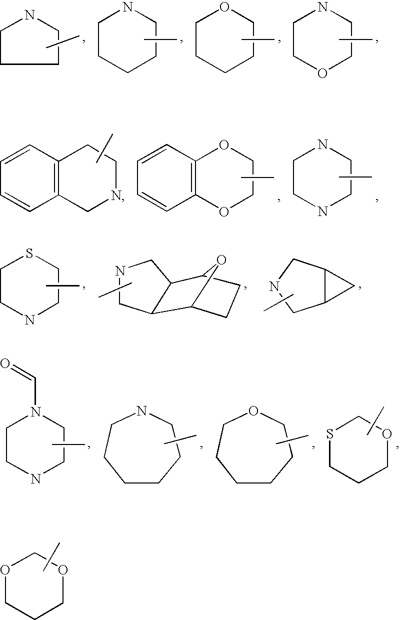
and the like. The above groups may include 1 to 4 substituents such as alkyl, halo, oxo and/or any of of the substituents for alkyl or aryl set out herein. In addition, any of the cycloheteroalkyl rings can be fused to a cycloalkyl, aryl, heteroaryl or cycloheteroalkyl ring.

Unless otherwise indicated, the term “heteroaryl” as used herein alone or as part of another group refers to a 5- or 6-membered aromatic ring including 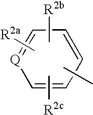
where Q is N, which includes 1, 2, 3 or 4 hetero atoms such as nitrogen, oxygen or sulfur, and such rings fused to an aryl, cycloalkyl, heteroaryl or cycloheteroalkyl ring (e.g. benzothiophenyl, indolyl), and includes possible N-oxides. The heteroaryl group may optionally include 1 to 4 substituents such as any of the the substituents for alkyl or aryl set out above. Examples of heteroaryl groups include the following: 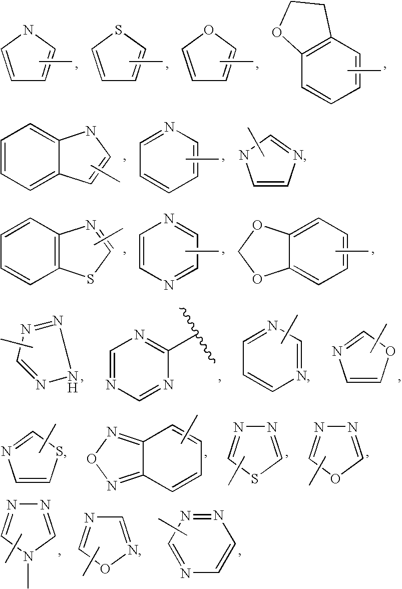
and the like.

The term “cycloheteroalkylalkyl” as used herein alone or as part of another group refers to cycloheteroalkyl groups as defined above linked through a C atom or heteroatom to a (CH₂)ₚ chain.

The term “heteroarylalkyl” or “heteroarylalkenyl” as used herein alone or as part of another group refers to a heteroaryl group as defined above linked through a C atom or heteroatom to a —(CH₂)ₚ— chain, alkylene or alkenylene as defined above.

The term “polyhaloalkyl” as used herein refers to an “alkyl” group as defined above which includes from 2 to 9, preferably from 2 to 5, halo substituents, such as F or Cl, preferably F, such as CF₃CH₂, CF₃ or CF₃CF₂CH₂.

The term “polyhaloalkyloxy” as used herein refers to an “alkoxy” or “alkyloxy” group as defined above which includes from 2 to 9, preferably from 2 to 5, halo substituents, such as F or Cl, preferably F, such as CF₃CH₂O, CF₃O or CF₃CF₂CH₂O.

The term “prodrug esters” as employed herein includes prodrug esters which are known in the art for carboxylic and phosphorus acid esters such as methyl, ethyl, benzyl and the like. Other prodrug ester examples of R⁴ include the following groups:
(1-alkanoyloxy)alkyl such as, 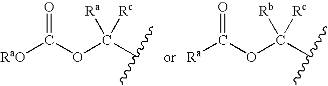
wherein R^{a}, R^{b} and R^{c} are H, alkyl, aryl or arylalkyl; however, R^{a}O cannot be HO.

Examples of such prodrug esters R⁴ include

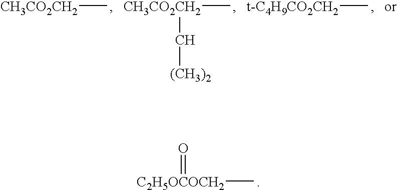

Other examples of suitable prodrug esters R⁴ include 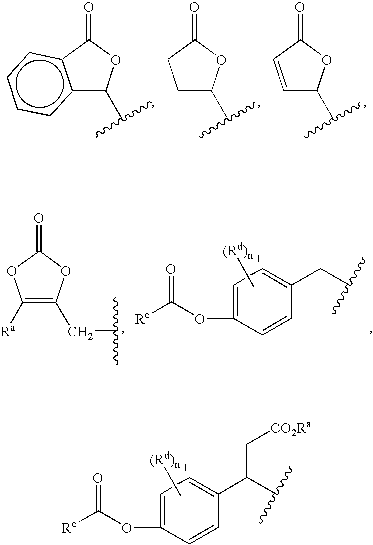
wherein R^{a} can be H, alkyl (such as methyl or t-butyl), arylalkyl (such as benzyl) or aryl (such as phenyl); R^{d} is H, alkyl, halogen or alkoxy, R^{e} is alkyl, aryl, arylalkyl or alkoxyl, and n, is 0, 1 or 2.

Where the compounds of Formula I are in acid form they may form a pharmaceutically acceptable salt such as alkali metal salts such as lithium, sodium or potassium, alkaline earth metal salts such as calcium or magnesium as well as zinc or aluminum and other cations such as ammonium, choline, diethanolamine, lysine (D or L), ethylenediamine, t-butylamine, t-octylamine, tris-(hydroxymethyl)aminomethane (TRIS), N-methyl glucosamine (NMG), triethanolamine and dehydroabietylamine.

All stereoisomers of the compounds of the instant invention are contemplated, either in admixture or in pure or substantially pure form. The compounds of the present invention can have chiral centers at any of the carbon atoms including any one of the R substituents. Consequently, compounds of Formula I can exist in enantiomeric or diastereomeric forms or in mixtures thereof. The processes for preparation can utilize racemates, enantiomers or diastereomers as starting materials. When diastereomeric or enantiomeric products are prepared, they can be separated by conventional methods for example, chromatographic or fractional crystallization.

The invention may be embodied in other specific forms without departing from the spirit or essential attributes thereof. This invention also encompasses all combinations of alternative aspects of the invention noted herein. It is understood that any and all embodiments of the present invention may be taken in conjunction with any other embodiment to describe additional embodiments of the present invention. Furthermore, any elements of an embodiment may be combined with any and all other elements from any of the embodiments to describe additional embodiments.

### Utilities and Combinations

Where desired, the compounds of Formula I may be used in combination with one or more anti-dyslipidemic agents or lipid-lowering agents and/or one or more other types of therapeutic agents including antidiabetic agents, anti-obesity agents, antihypertensive agents, platelet aggregation inhibitors, and/or anti-osteoporosis agents, which may be administered orally in the same dosage form, in a separate oral dosage form or by injection.

The anti-dyslipidemic agent or lipid-lowering agent which may be optionally employed in combination with the compounds of Formula I of the invention may include 1, 2, 3 or more MTP inhibitors, HMG CoA reductase inhibitors, squalene synthetase inhibitors, ACAT inhibitors, lipoxygenase inhibitors, cholesterol absorption inhibitors, ileal Na⁺/bile acid cotransporter inhibitors, upregulators of LDL receptor activity, bile acid sequestrants, cholesterol ester transfer protein (CETP) inhibitors [e.g., torcetrapib (Pfizer) and JTT-302 (Japan Tobacco)], and/or nicotinic acid and derivatives thereof.

MTP inhibitors employed herein include MTP inhibitors disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. application Ser. No. 09/175,180 filed Oct. 20, 1998, now U.S. Pat. No. 5,962,440. Preferred are each of the preferred MTP inhibitors disclosed in each of the above patents and applications.

All of the above U.S. Patents and applications are incorporated herein by reference.

Most preferred MTP inhibitors to be employed in accordance with the present invention include preferred MTP inhibitors as set out in U.S. Pat. Nos. 5,739,135 and 5,712,279, and U.S. Pat. No. 5,760,246.

The most preferred MTP inhibitor is 9-[4-[4-[[2-(2,2,2-Trifluoroethoxy)benzoyl]amino]-1-piperidinyl]butyl]-N-(2,2,2-trifluoroethyl)-9H-fluorene-9-carboxamide

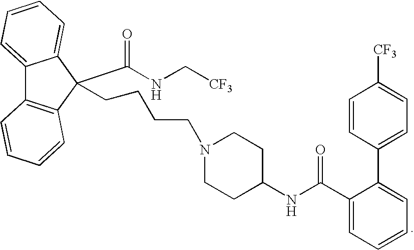

The anti-dyslipidemic agent may be an HMG CoA reductase inhibitor which includes, but is not limited to, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, atorvastatin disclosed in U.S. Pat. Nos. 4,681,893, 5,273,995, 5,385,929 and 5,686,104, itavastatin (Nissan/Sankyo's nisvastatin (NK-104)) disclosed in U.S. Pat. No. 5,011,930, Shionogi-Astra/Zeneca visastatin (ZD-4522) disclosed in U.S. Pat. No. 5,260,440, and related statin compounds disclosed in U.S. Pat. No. 5,753,675, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0,142,146 A2, and quinoline and pyridine derivatives disclosed in U.S. Pat. Nos. 5,506,219 and 5,691,322.

In addition, phosphinic acid compounds useful in inhibiting HMG CoA reductase suitable for use herein are disclosed in GB 2205837.

The squalene synthetase inhibitors suitable for use herein include, but are not limited to, α-phosphono-sulfonates disclosed in U.S. Pat. No. 5,712,396, those disclosed by Biller et al, J. Med. Chem., 1988, Vol. 31, No. 10, pp. 1869-1871, including isoprenoid (phosphinyl-methyl)phosphonates as well as other known squalene synthetase inhibitors, for example, as disclosed in U.S. Pat. No. 4,871,721 and 4,924,024 and in Biller, S. A., Neuenschwander, K., Ponpipom, M. M., and Poulter, C. D., Current Pharmaceutical Design, 2, 1-40 (1996).

In addition, other squalene synthetase inhibitors suitable for use herein include the terpenoid pyrophosphates disclosed by P. Ortiz de Montellano et al, J. Med. Chem., 1977, 20:243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109:5544 and cyclopropanes reported by Capson, T. L., PhD dissertation, June, 1987, Dept. Med. Chem. U of Utah, Abstract, Table of Contents, pp 16, 17, 40-43, 48-51, Summary.

Other anti-dyslipidemic agents suitable for use herein include, but are not limited to, probucol, and related compounds as disclosed in U.S. Pat. No. 3,674,836, probucol being preferred; bile acid sequestrants such as cholestyramine, colestipol and DEAE-Sephadex (Secholex®, Policexide®) and cholestagel (Sankyo/Geltex), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives); nicotinic acid (niacin), acipimox, acifran, neomycin, p-aminosalicylic acid, aspirin; poly(diallylmethylamine) derivatives such as disclosed in U.S. Pat. No. 4,759,923; quaternary amine poly(diallyldimethylammonium chloride) and ionenes such as disclosed in U.S. Pat. No. 4,027,009, and other known serum cholesterol lowering agents.

The anti-dyslipidemic agent may be an ACAT inhibitor such as disclosed in, Drugs of the Future 24, 9-15 (1999), (Avasimibe); “The ACAT inhibitor, C1-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent dyslipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for dyslipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62, or TS-962 (Taisho Pharmaceutical Co. Ltd).

The anti-dyslipidemic agent may be an upregulator of LD2 receptor activity such as MD-700 (Taisho Pharmaceutical Co. Ltd) and LY295427 (Eli Lilly).

The anti-dyslipidemic agent may be a cholesterol absorption inhibitor preferably Schering-Plough's SCH48461 as well as those disclosed in Atherosclerosis 115, 45-63 (1995) and J. Med. Chem. 41, 973 (1998).

The anti-dyslipidemic agent may be an ileal Na⁺/bile acid cotransporter inhibitor such as disclosed in Drugs of the Future, 24, 425-430 (1999).

The lipid-modulating agent may be a cholesteryl ester transfer protein (CETP) inhibitor such as Pfizer's CP 529,414 (WO/0038722 and EP 818448) and Pharmacia's SC-744 and SC-795.

The ATP citrate lyase inhibitor which may be employed in the combination of the invention may include, for example, those disclosed in U.S. Pat. No. 5,447,954.

Preferred anti-dyslipidemic agents are pravastatin, lovastatin, simvastatin, atorvastatin, fluvastatin, itavastatin and visastatin and ZD-4522.

The above-mentioned U.S. patents are incorporated herein by reference. The amounts and dosages employed will be as indicated in the Physician's Desk Reference and/or in the patents set out above.

The compounds of Formula I of the invention will be employed in a weight ratio to the anti-dyslipidemic agent (where present), within the range from about 500:1 to about 1:500, preferably from about 100:1 to about 1:100.

The dose administered must be carefully adjusted according to age, weight and condition of the patient, as well as the route of administration, dosage form and regimen and the desired result.

The dosages and formulations for the anti-dyslipidemic agent will be as disclosed in the various patents and applications discussed above.

The dosages and formulations for the other anti-dyslipidemic agent to be employed, where applicable, will be as set out in the latest edition of the Physicians' Desk Reference.

For oral administration, a satisfactory result may be obtained employing the MTP inhibitor in an amount within the range of from about 0.01 mg to about 500 mg and preferably from about 0.1 mg to about 100 mg, one to four times daily.

A preferred oral dosage form, such as tablets or capsules, will contain the MTP inhibitor in an amount of from about 1 to about 500 mg, preferably from about 2 to about 400 mg, and more preferably from about 5 to about 250 mg, one to four times daily.

For oral administration, a satisfactory result may be obtained employing an HMG CoA reductase inhibitor, for example, pravastatin, lovastatin, simvastatin, atorvastatin, fluvastatin or rosuvastatin in dosages employed as indicated in the Physician's Desk Reference, such as in an amount within the range of from about 1 to 2000 mg, and preferably from about 4 to about 200 mg.

The squalene synthetase inhibitor may be employed in dosages in an amount within the range of from about 10 mg to about 2000 mg and preferably from about 25 mg to about 200 mg.

A preferred oral dosage form, such as tablets or capsules, will contain the HMG CoA reductase inhibitor in an amount from about 0.1 to about 100 mg, preferably from about 0.5 to about 80 mg, and more preferably from about 1 to about 40 mg.

A preferred oral dosage form, such as tablets or capsules will contain the squalene synthetase inhibitor in an amount of from about 10 to about 500 mg, preferably from about 25 to about 200 mg.

The anti-dyslipidemic agent may also be a lipoxygenase inhibitor including a 15-lipoxygenase (15-LO) inhibitor such as benzimidazole derivatives as disclosed in WO 97/12615, 15-LO inhibitors as disclosed in WO 97/12613, isothiazolones as disclosed in WO 96/38144, and 15-LO inhibitors as disclosed by Sendobry et al “Attenuation of diet-induced atherosclerosis in rabbits with a highly selective 15-lipoxygenase inhibitor lacking significant antioxidant properties”, Brit. J. Pharmacology (1997) 120, 1199-1206, and Cornicelli et al, “15-Lipoxygenase and its Inhibition: A Novel Therapeutic Target for Vascular Disease”, Current Pharmaceutical Design, 1999, 5, 11-20.

The compounds of Formula I and the anti-dyslipidemic agent may be employed together in the same oral dosage form or in separate oral dosage forms taken at the same time.

The compositions described above may be administered in the dosage forms as described above in single or divided doses of one to four times daily. It may be advisable to start a patient on a low dose combination and work up gradually to a high dose combination.

The preferred anti-dyslipidemic agent is pravastatin, simvastatin, lovastatin, atorvastatin, fluvastatin or rosuvastatin as well as niacin and/or cholestagel.

The other antidiabetic agent which may be optionally employed in combination with the compound of Formula I may be 1, 2, 3 or more antidiabetic agents or antihyperglycemic agents including insulin secretagogues or insulin sensitizers, or other antidiabetic agents preferably having a mechanism of action different from the compounds of Formula I of the invention, which may include biguanides, sulfonyl ureas, glucosidase inhibitors, PPAR γ agonists, such as thiazolidinediones, dipeptidyl peptidase IV (DP4) inhibitors, SGLT2 inhibitors, and/or meglitinides, as well as insulin, and/or glucagon-like peptide-1 (GLP-1).

The other antidiabetic agent may be an oral antihyperglycemic agent preferably a biguanide such as metformin or phenformin or salts thereof, preferably metformin HCl.

Where the antidiabetic agent is a biguanide, the compounds of Formula I will be employed in a weight ratio to biguanide within the range from about 0.001:1 to about 10:1, preferably from about 0.01:1 to about 5:1.

The other antidiabetic agent may also preferably be a sulfonyl urea such as glyburide (also known as glibenclamide), glimepiride (disclosed in U.S. Pat. No. 4,379,785), glipizide, gliclazide or chlorpropamide, other known sulfonylureas or other antihyperglycemic agents which act on the ATP-dependent channel of the β-cells, with glyburide and glipizide being preferred, which may be administered in the same or in separate oral dosage forms.

The compounds of Formula I will be employed in a weight ratio to the sulfonyl urea in the range from about 0.01:1 to about 100:1, preferably from about 0.02:1 to about 5:1.

The oral antidiabetic agent may also be a glucosidase inhibitor such as acarbose (disclosed in U.S. Pat. No. 4,904,769) or miglitol (disclosed in U.S. Pat. No. 4,639,436), which may be administered in the same or in a separate oral dosage forms.

The compounds of Formula I will be employed in a weight ratio to the glucosidase inhibitor within the range from about 0.01:1 to about 100:1, preferably from about 0.05:1 to about 10:1.

The compounds of Formula I may be employed in combination with a PPAR γ agonist such as a thiazolidinedione oral anti-diabetic agent or other insulin sensitizers (which has an insulin sensitivity effect in NIDDM patients) such as rosiglitazone (SKB), pioglitazone (Takeda), Mitsubishi's MCC-555 (disclosed in U.S. Pat. No. 5,594,016), R-119702 (Sankyo/WL), or YM-440 (Yamanouchi), preferably rosiglitazone and pioglitazone.

The compounds of Formula I will be employed in a weight ratio to the thiazolidinedione in an amount within the range from about 0.01:1 to about 100:1, preferably from about 0.05 to about 10:1.

The sulfonyl urea and thiazolidinedione in amounts of less than about 150 mg oral antidiabetic agent may be incorporated in a single tablet with the compounds of Formula I.

The compounds of Formula I may also be employed in combination with a antihyperglycemic agent such as insulin or with glucagon-like peptide-1 (GLP-1) such as GLP-1(1-36) amide, GLP-1(7-36) amide, GLP-1(7-37) (as disclosed in U.S. Pat. No. 5,614,492 to Habener, the disclosure of which is incorporated herein by reference), as well as AC2993 (Amylin) and LY-315902 (Lilly), which may be administered via injection, intranasal, inhalation or by transdermal or buccal devices.

Where present, metformin, the sulfonyl ureas, such as glyburide, glimepiride, glipyride, glipizide, chlorpropamide and gliclazide and the glucosidase inhibitors acarbose or miglitol or insulin (injectable, pulmonary, buccal, or oral) may be employed in formulations as described above and in amounts and dosing as indicated in the Physician's Desk Reference (PDR).

Where present, metformin or salt thereof may be employed in amounts within the range from about 500 to about 2000 mg per day which may be administered in single or divided doses one to four times daily.

Where present, the thiazolidinedione anti-diabetic agent may be employed in amounts within the range from about 0.01 to about 2000 mg/day which may be administered in single or divided doses one to four times per day.

Where present insulin may be employed in formulations, amounts and dosing as indicated by the Physician's Desk Reference.

Where present GLP-1 peptides may be administered in oral buccal formulations, by nasal administration or parenterally as described in U.S. Pat. Nos. 5,346,701 (TheraTech), 5,614,492 and 5,631,224 which are incorporated herein by reference.

The other antidiabetic agent may also be a PPAR α/γ dual agonist such as Muraglitazar (Bristol-Myers Squibb).

The antidiabetic agent may be an SGLT2 inhibitor such as disclosed in U.S. Pat. No. 6,414,126, employing dosages as set out therein. Preferred are the compounds designated as preferred in the above patent. Other suitable SGLT2 inhibitors include T-1095, phlorizin, WAY-123783, and those described in WO 01/27128, U.S. Pat. No. 6,515,117 and U.S. Pat. No. 6,414,126.

The antidiabetic agent may be a DPP4 inhibitor. These include saxagliptin (Bristol-Myers Squibb), vildagliptin (Novartis), sitagliptin (Merck) and alogliptin (Takeda) as well as those such as disclosed in WO99/38501, WO99/46272, WO99/67279 (PROBIODRUG), WO99/67278 (PROBIODRUG), WO99/61431 (PROBIODRUG), NVP-DPP728A (1-[[[2-[(5-cyanopyridin-2-yl)amino]ethyl]amino]acetyl]-2-cyano-(S)-pyrrolidine) (Novartis) as disclosed by Hughes et al, Biochemistry, 38(36), 11597-11603, 1999, TSL-225 (tryptophyl-1,2,3,4-tetrahydro-isoquinoline-3-carboxylic acid) disclosed by Yamada et al, Bioorg. & Med. Chem. Lett. 8 (1998) 1537-1540, 2-cyanopyrrolidides and 4-cyanopyrrolidides as disclosed by Ashworth et al, Bioorg. & Med. Chem. Lett., Vol. 6, No. 22, pp 1163-1166 and 2745-2748 (1996) employing dosages as set out in the above references.

The meglitinide which may optionally be employed in combination with the compound of Formula I of the invention may be repaglinide, nateglinide (Novartis) or KAD1229 (PF/Kissei), with repaglinide being preferred.

The compound of Formula I will be employed in a weight ratio to the meglitinide, PPAR-α/γ dual agonist, DP4 inhibitor or SGLT2 inhibitor within the range from about 0.01:1 to about 100:1, preferably from about 0.05 to about 10:1.

The other type of therapeutic agent which may be optionally employed with a compound of Formula I may be 1, 2, 3 or more of an anti-obesity agent including a beta 3 adrenergic agonist, a lipase inhibitor, a serotonin (and dopamine) reuptake inhibitor, a thyroid receptor agonist, a cannabinoid receptor 1 (CB-1) antagonist and/or an anorectic agent.

The beta 3 adrenergic agonist which may be optionally employed in combination with a compound of Formula I may be AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, with AJ9677, L750,355 and CP331648 being preferred.

The lipase inhibitor which may be optionally employed in combination with a compound of Formula I may be orlistat or ATL-962 (Alizyme), with orlistat being preferred.

The serotonin (and dopamine) reuptake inhibitor which may be optionally employed in combination with a compound of Formula I may be sibutramine, topiramate (Johnson & Johnson) or axokine (Regeneron), with sibutramine and topiramate being preferred.

The thyroid receptor agonist which may be optionally employed in combination with a compound of Formula I may be a thyroid receptor ligand as disclosed in WO97/21993 (U. Cal SF), WO99/00353 (KaroBio), WO 00/039077 (KaroBio), and U.S. Pat. No. 6,800,605, with compounds of the KaroBio applications and the above patent being preferred.

Cannabinoid receptor 1 antagonists and inverse agonists which may be optionally employed in combination with compounds of the present invention include rimonabant, SLV 319, and those discussed in D. L. Hertzog, Expert Opin. Ther. Patents 2004, 14, 1435-1452.

The anorectic agent which may be optionally employed in combination with a compound of Formula I may be dexamphetamine, phentermine, phenylpropanolamine or mazindol, with dexamphetamine being preferred.

The various anti-obesity agents described above may be employed in the same dosage form with the compound of Formula I or in different dosage forms, in dosages and regimens as generally known in the art or in the PDR.

The antihypertensive agents which may be employed in combination with the compound of Formula I of the invention include ACE inhibitors, angiotensin II receptor antagonists, NEP/ACE inhibitors, as well as calcium channel blockers, β-adrenergic blockers and other types of antihypertensive agents, including diuretics.

The angiotensin converting enzyme inhibitor which may be employed herein includes those containing a mercapto (—S—) moiety such as substituted proline derivatives, such as any of those disclosed in U.S. Pat. No. 4,046,889 to Ondetti et al mentioned above, with captopril, that is, 1-[(2S)-3-mercapto-2-methylpropionyl]-L-proline, being preferred, and mercaptoacyl derivatives of substituted prolines such as any of those disclosed in U.S. Pat. No. 4,316,906 with zofenopril being preferred.

Other examples of mercapto containing ACE inhibitors that may be employed herein include rentiapril (fentiapril, Santen) disclosed in Clin. Exp. Pharmacol. Physiol. 10:131 (1983); as well as pivopril and YS980.

Other examples of angiotensin converting enzyme inhibitors which may be employed herein include any of those disclosed in U.S. Pat. No. 4,374,829 mentioned above, with N-(1-ethoxycarbonyl-3-phenylpropyl)-L-alanyl-L-proline, that is, enalapril, being preferred; any of the phosphonate substituted amino or imino acids or salts disclosed in U.S. Pat. No. 4,452,790, with (S)-1-[6-amino-2-[[hydroxy-(4-phenylbutyl)phosphinyl]oxy]-1-oxohexyl]-L-proline or (ceronapril) being preferred; phosphinylalkanoyl prolines disclosed in U.S. Pat. No. 4,168,267 mentioned above with fosinopril being preferred; any of the phosphinylalkanoyl substituted prolines disclosed in U.S. Pat. No. 4,337,201; and the phosphonamidates disclosed in U.S. Pat. No. 4,432,971 discussed above.

Other examples of ACE inhibitors that may be employed herein include Beecham's BRL 36,378 as disclosed in European Patent Application Nos. 80822 and 60668; Chugai's MC-838 disclosed in C.A. 102:72588v and Jap. J. Pharmacol. 40:373 (1986); Ciba-Geigy's CGS 14824 (3-([1-ethoxycarbonyl-3-phenyl-(1S)-propyl]amino)-2,3,4,5-tetrahydro-2-oxo-1-(3S)-benzazepine-1 acetic acid HCl) disclosed in U.K. Patent No. 2103614 and CGS 16,617 (3(S)-[[(1S)-5-amino-1-carboxypentyl]amino]-2,3,4,5-tetrahydro-2-oxo-1H-1-benzazepine-1-ethanoic acid) disclosed in U.S. Pat. No. 4,473,575; cetapril (alacepril, Dainippon) disclosed in Eur. Therap. Res. 39:671 (1986); ramipril (Hoechst) disclosed in Euro. Patent No. 79-022 and Curr. Ther. Res. 40:74 (1986); Ru 44570 (Hoechst) disclosed in Arzneimittelforschung 34:1254 (1985); cilazapril (Hoffman-LaRoche) disclosed in J. Cardiovasc. Pharmacol. 9:39 (1987); R 31-2201 (Hoffman-LaRoche) disclosed in FEBS Lett. 165:201 (1984); lisinopril (Merck); indalapril (delapril) disclosed in U.S. Pat. No. 4,385,051; indolapril (Schering) disclosed in J. Cardiovasc. Pharmacol. 5:643, 655 (1983); spirapril (Schering) disclosed in Acta. Pharmacol. Toxicol. 59 (Supp. 5):173 (1986); perindopril (Servier) disclosed in Eur. J. Clin. Pharmacol. 31:519 (1987); quinapril (Warner-Lambert) disclosed in U.S. Pat. No. 4,344,949 and C1925 (Warner-Lambert) ([3S-[2[R(*)R(*)]]3R(*)]-2-[2-[[1-(ethoxy-carbonyl)-3-phenylpropyl]amino]-1-oxopropyl]-1,2,3,4-tetrahydro-6,7-dimethoxy-3-isoquinolinecarboxylic acid HCl) disclosed in Pharmacologist 26:243, 266 (1984), WY-44221 (Wyeth) disclosed in J. Med. Chem. 26:394 (1983).

Preferred ACE inhibitors are captopril, fosinopril, enalapril, lisinopril, quinapril, benazepril, fentiapril, ramipril and moexipril.

NEP/ACE inhibitors may also be employed herein in that they possess neutral endopeptidase (NEP) inhibitory activity and angiotensin converting enzyme (ACE) inhibitory activity. Examples of NEP/ACE inhibitors suitable for use herein include those disclosed in U.S. Pat. Nos. 5,362,727, 5,366,973, 5,225,401, 4,722,810, 5,223,516, 4,749,688, 5,552,397, 5,504,080, 5,612,359, and 5,525,723, European Patent Applications 0599,444, 0481,522, 0599,444, 0595,610, 0534363A2, 534,396, 534,492, and 0629627A2.

Preferred are those NEP/ACE inhibitors and dosages thereof which are designated as preferred in the above patents/applications which U.S. patents are incorporated herein by reference; most preferred are omapatrilat, BMS 189,921 ([S-(R*,R*)]-hexahydro-6-[(2-mercapto-1-oxo-3-phenylpropyl)amino]-2,2-dimethyl-7-oxo-1H-azepine-1-acetic acid (gemopatrilat)) and CGS 30440.

The angiotensin II receptor antagonist (also referred to herein as angiotensin II antagonist or AII antagonist) suitable for use herein includes, but is not limited to, irbesartan, losartan, valsartan, candesartan, telmisartan, tasosartan or eprosartan, with irbesartan, losartan or valsartan being preferred.

A preferred oral dosage form, such as tablets or capsules, will contain the ACE inhibitor or AII antagonist in an amount within the range from abut 0.1 to about 500 mg, preferably from about 5 to about 200 mg and more preferably from about 10 to about 150 mg.

For parenteral administration, the ACE inhibitor, angiotensin II antagonist or NEP/ACE inhibitor will be employed in an amount within the range from about 0.005 mg/kg to about 10 mg/kg and preferably from about 0.01 mg/kg to about 1 mg/kg.

Where a drug is to be administered intravenously, it will be formulated in conventional vehicles, such as distilled water, saline, Ringer's solution or other conventional carriers.

It will be appreciated that preferred dosages of ACE inhibitor and AII antagonist as well as other antihypertensives disclosed herein will be as set out in the latest edition of the Physician's Desk Reference (PDR).

Other examples of preferred antihypertensive agents suitable for use herein include omapatrilat (Vanlev®), amlodipine besylate (Norvasc®), prazosin HCl (Minipress®), verapamil, nifedipine, nadolol, diltiazem, felodipine, nisoldipine, isradipine, nicardipine, atenolol, carvedilol, sotalol, terazosin, doxazosin, propranolol, and clonidine HCl (Catapres®).

Diuretics which may be employed in combination with compounds of Formula I include hydrochlorothiazide, torasemide, furosemide, spironolactono, and indapamide.

Antiplatelet agents which may be employed in combination with compounds of Formula I of the invention include aspirin, clopidogrel, ticlopidine, dipyridamole, abciximab, tirofiban, eptifibatide, anagrelide, and ifetroban, with clopidogrel and aspirin being preferred.

The antiplatelet drugs may be employed in amounts as indicated in the PDR. Ifetroban may be employed in amounts as set out in U.S. Pat. No. 5,100,889.

Antiosteoporosis agents suitable for use herein in combination with the compounds of Formula I of the invention include parathyroid hormone or bisphosphonates, such as MK-217 (alendronate) (Fosamax®). Dosages employed will be as set out in the PDR.

In carrying out the method of the invention, a pharmaceutical composition will be employed containing the compounds of Formula I, with or without another therapeutic agent, in association with a pharmaceutical vehicle or diluent. The pharmaceutical composition can be formulated employing conventional solid or liquid vehicles or diluents and pharmaceutical additives of a type appropriate to the mode of desired administration. The compounds can be administered to mammalian species including humans, monkeys, dogs, etc. by an oral route, for example, in the form of tablets, capsules, granules or powders, or they can be administered by a parenteral route in the form of injectable preparations. The dose for adults is preferably between 0.1 and 2,000 mg per day, which can be administered in a single dose or in the form of individual doses from 1-4 times per day. Alternatively, another preferred mode of administration may be intermittent dosing (i.e., a single dose of drug administered at intervals, ranging from once every 2 days to once every 7 days).

A typical capsule for oral administration contains compounds of Formula I (25 mg), lactose (7.5 mg) and magnesium stearate (1.5 mg). The mixture is passed through a 60 mesh sieve and packed into a No. 1 gelatin capsule.

The following Examples represent preferred embodiments of the invention.

### ABBREVIATIONS

For ease of reference, the following abbreviations are employed herein, including the methods of preparation and Examples that follow:
aq.=aqueous
Ar=argon
Bn=benzyl
Boc=tert-butoxycarbonyl
BOP reagent=benzotriazol-1-yloxy-tris(dimethylamino)phosphonium hexafluorophosphate
CAN=ceric ammonium nitrate
Cbz=carbobenzyloxy or carbobenzoxy or benzyloxycarbonyl
Cbz-Cl=benzyl chloroformate
DBU=1,8-diazabicyclo[5.4.0]undec-7-ene
DCE=1,2 dichloroethane
DEAD=diethyl azodicarboxylate
DIAD=diisopropyl azodicarboxylate
DIBALH=diisobutyl aluminum hydride
DMAP=4-dimethylaminopyridine
DME=1,2 dimethoxyethane
DMF=dimethyl formamide
DMSO=dimethyl sulfoxide
EDC (or EDC.HCl) or EDCI (or EDCI.HCl) or EDAC=3-ethyl-3′-(dimethylamino)propyl-carbodiimide hydrochloride (or 1-(3-dimethylaminopropyl)-3-ethylcarbodiimide hydrochloride)
Et=ethyl
Et₂O=diethyl ether
Et₃N=triethylamine
EtOAc=ethyl acetate
EtOH=ethanol
FMOC=fluorenylmethoxycarbonyl
g=gram(s)
h or hr=hour(s)
hex=hexanes
HMPA=hexamethyl phosphoric triamide
HOAc or AcOH=acetic acid
HOAT=1-Hydroxy-7-azabenzotriazole
HOBT or HOBT.H₂O=1-hydroxybenzotriazole hydrate
HPLC=high performance liquid chromatography
i-Pr₂NEt=diisopropylethylamine
i-PrOH═IPA=isopropanol
K₂CO₃=potassium carbonate
KOH=potassium hydroxide
L=liter
LC/MS=high performance liquid chromatography/mass spectrometry
LiAlH₄=lithium aluminum hydride
LiOH=lithium hydroxide
LRMS=low resolution mass spectrometry
Me=methyl
MeOH=methanol
meq=milliequivalent
mg=milligram(s)
min=minute(s)
mL=milliliter
mmol=millimole(s)
mol=moles
mp=melting point
MS or Mass Spec=mass spectrometry
N₂=nitrogen
NaBH(OAc)₃=sodium triacetoxyborohydride
NaBH₄=sodium borohydride
NaHCO₃=sodium bicarbonate
NaN(TMS)₂=sodium hexamethyldisilazide or sodium bis(trimethylsilyl)amide
NaOH=sodium hydroxide
n-BuLi=n-butyllithium
NMM=N-methyl morpholine
NMR=nuclear magnetic resonance
NMR spectral data: s=singlet; d=doublet; m=multiplet; br=broad; t=triplet
Pd(OAc)₂=Palladium acetate
Pd/C=palladium on carbon
Ph=phenyl
Ph₃P=triphenylphosphine
(Ph₃P)₄Pd^{o}=tetrakis triphenylphosphine palladium
PtO₂=platinum oxide
RT=room temperature
sat or sat'd=saturated
SAX=Strong Anion Exchanger
SCX=Strong Cation Exchanger
TBS=tert-butyldimethylsilyl
t-Bu=tertiary butyl
TFA=trifluoroacetic acid
THF=tetrahydrofuran
TLC=thin layer chromatography
TMS=trimethylsilyl
TMSN₃=trimethylsilyl azide
μL=microliter

### Methods of Preparation

Schemes 1-2 describe a general synthetic sequence for the preparation of the compounds of Formula I. During the preparation of compounds of Formula I, one or more protecting groups might be used; reaction conditions for protection and deprotection may be found in the “Protective Groups in Organic Synthesis”, 3^{rd} Edition, T. W. Greene and P. G. M. Wuts, John Wiley and Sons Inc, 1999, or other methods used by one of ordinary skill in the art.

The compounds of Formula (I) may generally be prepared according to the following schemes and the knowledge of one skilled in the art.

### Schemes

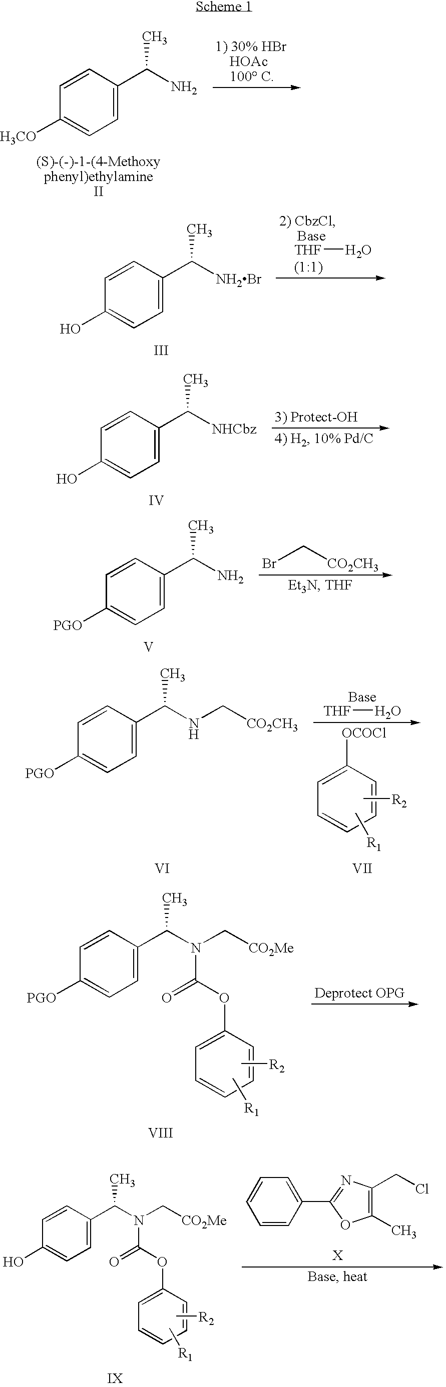

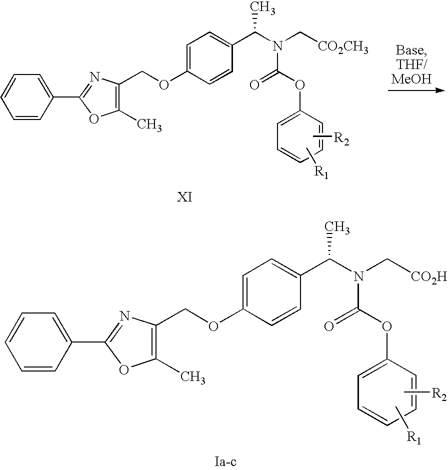

The synthesis of Compound I (including Compounds Ia, Ib and Ic) can be accomplished via the general route shown above in Scheme 1 which adapted the key steps from the previous synthesis of Example 498 in U.S. Pat. No. 6,414,002 B1, incorporated by reference herein. The synthesis of the key chiral secondary amine VI was accomplished using the 5-step sequence shown in the scheme below starting from (S)-(−)-1-(4-methoxyphenyl)ethylamine II. Demethylation of II with HBr in acetic acid with heating furnished the crude phenol-amine hydrobromide III, which was immediately reacted with benzyl chloroformate in aqueous base/THF to give the phenol-benzyl carbamate IV. Phenol IV was protected (e.g. as a tert-butyl dimethylsilyl ether) and the benzyl carbamate was subsequently hydrogenolyzed to provide the protected benzylamine V. Alkylation of amine V with methyl bromoacetate in the presence of base (e.g. triethylamine) furnished the key secondary amine intermediate VI. Acylation of secondary amine VI with an appropriately substituted aryl chloroformate VII under Schotten-Baumann conditions provided the carbamate-ester VIII, which was then deprotected to provide the aryl carbamate phenol IX. Alkylation of phenol IX with the chlorophenyloxazole X under basic conditions with heating gave the penultimate intermediate phenyloxazole carbamate ester XI. Hydrolysis of XI in aqueous lithium hydroxide/THF furnished Compounds Ia-Ic.

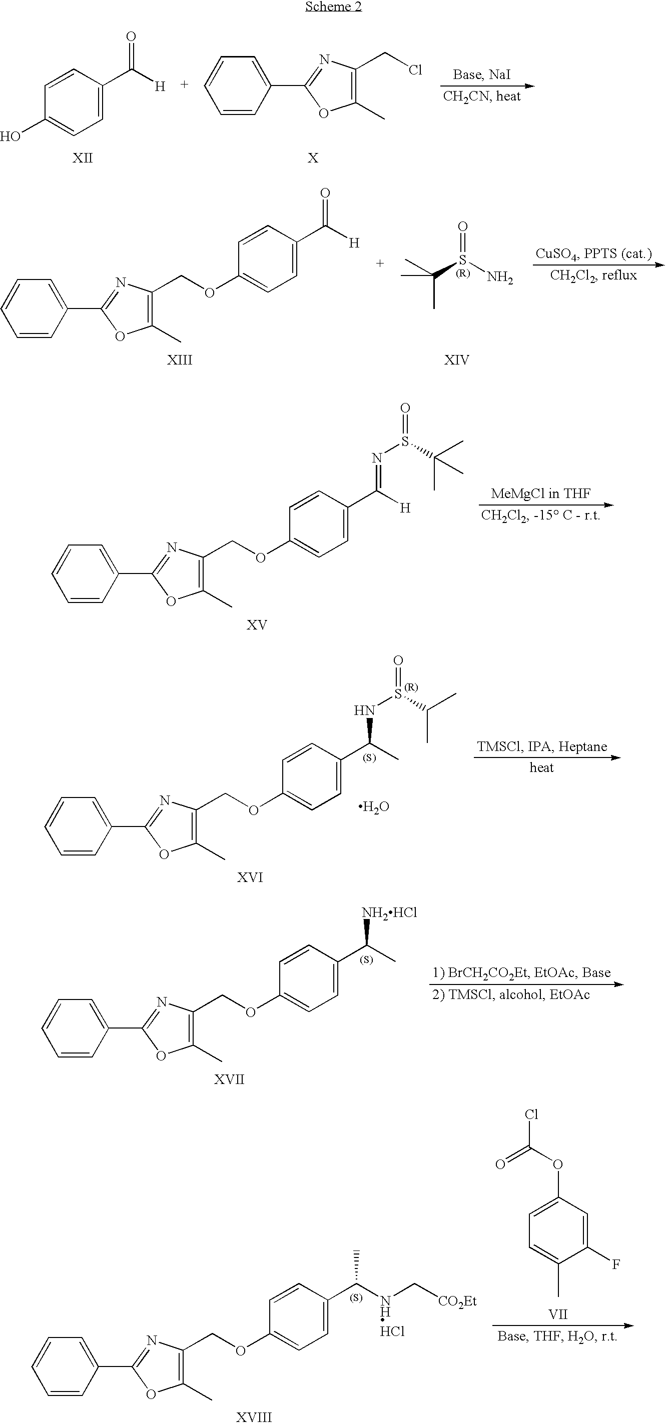

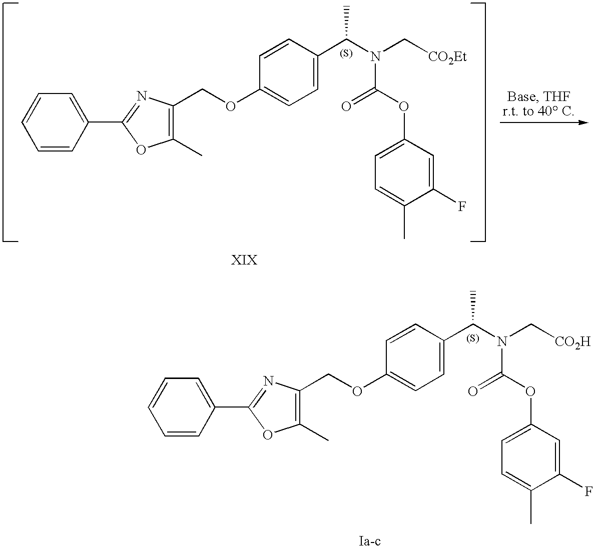

An alternative synthetic route to Compounds Ia-Ic is shown in Scheme 2 above. 4-hydroxybenzaldehyde XII is alkylated with chlorophenyl oxazole X in the presence of base and sodium iodide to give the alkylated phenyloxazole-benzaldehyde XIII. Condensation of the chiral tert-butyl sulfinamide XIV furnishes the chiral imine XV. Highly diastereoselective addition of methyl magnesium bromide to the chiral imine XV provides the methylated sulfinamide XVI. The tert-butyl sulfonamide is deprotected to give the chiral α-methyl benzylamine XVII, which is alkylated with ethyl bromoacetate in the presence of base (e.g. triethylamine) to furnish the key secondary amine XVIII. Amine XVIII is acylated with an appropriate aryl chloroformate VII in the presence of aqueous base (e.g. Schotten-Baumann conditions) to give the carbamate esters XIX. Base-mediated hydrolysis of the esters XIX provides the desired compound Ia-Ic.

### EXAMPLES

### Example 1

### Preparation of Compound Ia (Procedure 1)

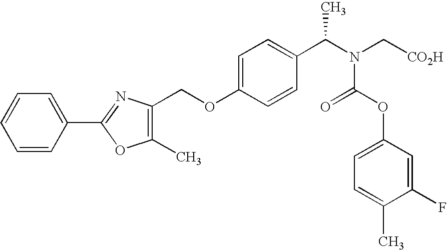

### Synthesis of 3-fluoro-4-methylphenyl carbonochloridate (2)

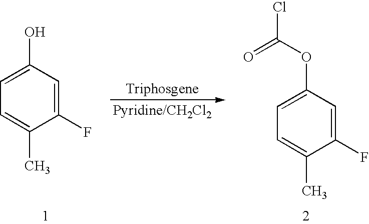

A 1 L 3-necked round bottom flask equipped with a mechanic stirrer was charged with 3-fluoro-4-methylphenol 1 (50 g, 446 mmol), triphosgene (43.67 g, 147 mmol) and CH₂Cl₂ (400 mL). Pyridine (35.2 g, 446 mmol) was added dropwise at 0° C. under N₂ over a period of 30 minutes. The reaction mixture was allowed to warm to room temperature over 45 minutes, and then stirred at reflux for an additional 2 hours. The reaction was cooled to room temperature, and the solvents were removed in vacuo. Anhydrous Et₂O (200 mL) was added to the residue, and the resulting slurry was filtered. The filtrate was concentrated in vacuo to afford crude 3-fluoro-4-methylphenyl chloroformate (83.5 g, 99% yield) as a thick oil, which was immediately used in the next step without further purification.

### Synthesis of 4-(chloromethyl)-5-methyl-2-phenyloxazole (3)

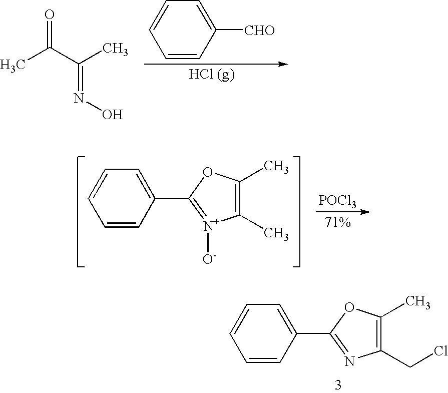

Gaseous HCl was bubbled through a mixture of benzaldehyde (110 g; 1.04 mol) and 2,3-butanedione monoxime (100 g, 0.990 mol) in EtOAc (500 mL) at 0° C. for ˜10 minutes. The reaction mixture was stirred overnight at room temperature and concentrated in vacuo (to 100 mL). A precipitate formed, which was filtered and washed with cold EtOAc. The crude N-oxide was dissolved in CHCl₃ (800 mL), and POCl₃ (101 mL, 1.09 mol) was added at room temperature in one portion. The reaction was heated to 60° C. and stirred at 60° C. for 12 hours, then cooled to room temperature and concentrated in vacuo. The residue was taken up in EtOAc and neutralized/basified with saturated aqueous NaHCO₃ and solid Na₂CO₃. The mixture was stirred vigorously for 15 min at room temperature and partitioned. The organic phase was dried (MgSO₄) and concentrated in vacuo to give the crude product (162 g) as a gray solid. This material was recrystallized from EtOAc/hexane to give 3 (109 g; 54%; HPLC purity=98.1%) as an off-white crystalline solid. An additional 47 g of crude product in the mother liquor was not further purified. A purified sample was characterized:

¹H NMR (CDCl₃, 400 MHz): δ 2.41 (s, 3H, CH₃), 4.56 (s, 2H, CH₂), 7.43 (m, 3H, Ar—H), 8.00 (m, 2H, Ar—H).

¹³C NMR (400 MHz, CDCl₃): δ 10.2 (CH₃), 37.2 (CH₂), 126.1 (CH), 127.1 (C), 128.6 (CH), 130.1 (CH), 132.8 (C), 146.5 (C), 159.9 (C).

LC/MS m/e 207 (M⁺); HPLC (continuous gradient from 50:50 Solvent A:B to 100% Solvent B over 8 min at 2.5 mL/min, where solvent A=90:10:0.2 H₂O:MeOH:H₃PO₄, and solvent B=90:10:0.2 MeOH:H₂O:H₃PO₄; Zorbax SB-C18 4.6×75 mm column, retention time=2.69 min.

### Synthesis of (S)-(−)-1-(4-hydroxyphenyl)ethylamine hydrobromide (6)

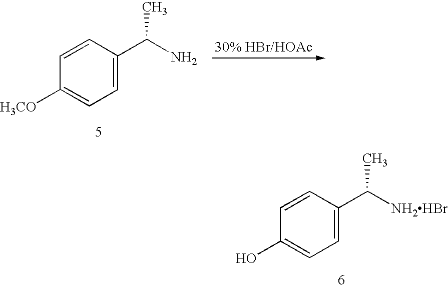

A solution of (S)-(−)-1-(4-methoxyphenyl)ethylamine (5, 75 g, 496.7 mmol) in 30% hydrogen bromide in acetic acid (350 mL, Aldrich) in a 1 liter sealed tube was heated at 100-110° C. in an oil bath for 6 hours. The reaction was cooled to room temperature, and nitrogen gas was bubbled into the solution to purge the excess HBr (the HBr was purged into aqueous NaOH). Volatiles were then removed in vacuo to afford the crude phenol amine HBr salt 9 (110 g) as a brown oil, which was used in the next reaction without further purification.

### Synthesis of (S)-(−)-1-(4-methoxyphenyl)ethylamine benzyl carbamate (7)

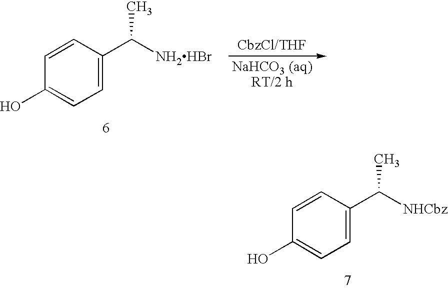

To a stirred room temperature solution of crude amine hydrobromide 6 (320 g) in 50% aqueous THF (400 mL) was slowly added solid NaHCO₃ (740 g; 6 equivalents) until the pH=8. Benzyl chloroformate (325 g, 1.9 mol) was added dropwise to the mixture, and the reaction was stirred at room temperature for 2 hours (until TLC indicated that the reaction was complete). The mixture was extracted with EtOAc (5×1 L), and the combined organic extracts were dried (Na₂SO₄) and concentrated in vacuo to afford crude compound 7 (520 g) as a brown oil, which was taken on to the next step without further purification.

A purified sample was characterized:

¹H NMR (400 MHz, CDCl₃): δ 1.07 (d, J=4.0 Hz, 3H, —CHCH₃—), 4.79 (m, 1H), 5.10 (m, 2H), 5.28 (d, J=8.0 Hz, 1H), 6.75 (d, J=4.0 Hz, 2H), 7.11 (d, J=4 Hz, 2H), 7.25-7.34 (m, 5H).

¹³C NMR (100 MHz, CDCl₃): δ 22.34, 50.21, 66.89, 115.44, 127.05, 127.59, 128.06, 128.42, 134.79, 136.07, 155.34, 155.86.

CHN: Calculated for C₁₆H₁₇NO₃: C, 70.83; H, 6.31; N, 5.16; Found: C, 70.78; H, 6.31; N, 5.07.

IR (KBr): 3316 (s, br), 1700 (s), 1686 (s), 1676 (s), 1530 (s), 1515 (s), 1259 (s), 1237 (s) cm⁻¹.

UV (MeOH; 16.4 mg/L): λₘₐₓ=283, 277 nm

[α]_{D}=−57.57° (c=10.1 mg/mL, MeOH, temperature=25° C.)

LRMS (M+Na⁺): 294.1; HRMS: Calculated for C₁₆H₁₇NO₃Na: 294.1106, found: 294.1118.

### Synthesis of (S)-benzyl 1-(4-(tert-butyldimethylsilyloxy)phenyl) ethylcarbamate (7^{a})

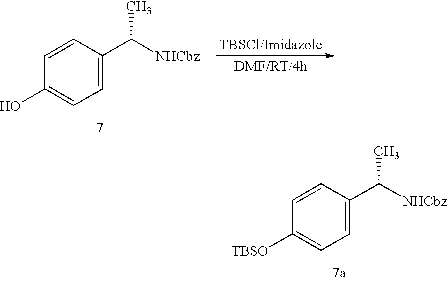

Imidazole (255 g, 3.75 mol) was added to a 0° C. solution of crude phenol carbamate 7 (510 g; 1.87 mol) in DMF (1.1 L). The mixture was stirred for 15 min, after which t-butyldimethylsilyl chloride (340 g; 2.7 mol) was added. The reaction was allowed to warm to RT and stirred for 1 h at RT, at which point TLC indicated that the reaction was complete. The solution was concentrated in vacuo, and the residue was partitioned between EtOAc (3 L) and aqueous 1.5 N aqueous HCl (1 L). The organic phase was washed with aqueous 1.5 N HCl (1 L), brine, dried (MgSO₄) and concentrated in vacuo. The crude product was chromatographed (SiO₂; 60-120 mesh, 9:1 hexane:EtOAc) to afford 7a (250 g of product which was 75% pure by ¹HNMR; the major impurity was TBSCl).

A purified sample was characterized:

Chiral HPLC (Daicel Chiralcel AD 4.6×250 mm column, isocratic 2% iPrOH/Heptane system, 30 min run) showed an ee of 100% (the retention time of the desired product is 13.9 min, while its enantiomer has a retention time of 11.6 min).

¹H NMR (400 MHz, CDCl₃): δ 7.26-7.42 (m, 5 H), 7.15 (d, J=7.83 Hz, 2 H), 6.78 (d, J=8.31 Hz, 2 H), 5.01-5.16 (m, 2 H), 4.94 (br. s., 1 H), 4.81 (br. s., 1 H), 1.46 (d, J=6.60 Hz, 3 H), 0.98 (s, 9 H), 0.18 (s, 6 H)

¹³C NMR (101 MHz, CDCl₃): δ 155.50, 154.88, 136.57, 136.09, 128.47, 128.05, 127.05, 120.05, 66.66, 50.17, 25.67, 22.30, 18.18, −4.42

[α]_{D}=−36.89° (c=3.79 mg/mL, CH₂Cl₂, temperature=25.1° C.)

LRMS (M+Na)⁺: 408.1

### Synthesis of (S)-1-(4-(tert-butyldimethylsilyloxy)phenyl)ethanamine (8)

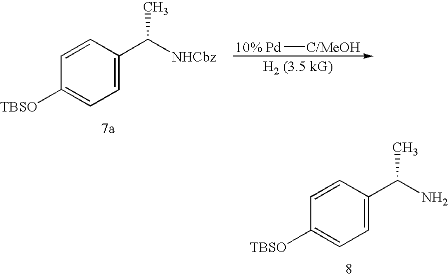

To a solution of partially purified 7a (250 g, 0.64 mol) in methanol (2.5 L) was cautiously added 10% palladium on carbon (25 g) under an atmosphere of nitrogen. The reaction mixture was subjected to hydrogenation under pressure (3.5 kG). After the reaction was complete, the mixture was filtered through Celite® and volatiles were removed in vacuo to give a semi-solid. This material was dissolved in CH₂Cl₂ (1.0 L), dried (anhydrous K₂CO₃) and concentrated in vacuo to give amine 8 as a yellow oil (175 g, 70%) which was sufficiently pure (95%) to be used in the next step without further purification.

¹H NMR (300 MHz; CDCl₃): δ 0.19 (s, 6H, Si—CH₃), 0.98 (s, 9H, Si-tBu), 1.40 (d, 3H, J=6.0 Hz, CH₃), 3.08 (s, 2H, NH₂), 4.04-4.10 (q, 1H, J=6 Hz, CH), 6.80-6.72 (d, 2H, J=9 Hz, Ar), 7.27-7.15 (d, 2H, J=9 Hz, Ar).

### Synthesis of (S)-methyl 2-(1-(4-(tert-butyldimethylsilyloxy)phenyl)ethylamino)acetate (4)

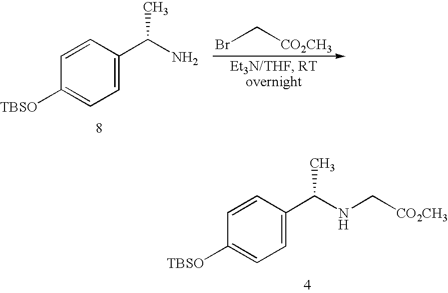

To a stirred solution of crude 8 (200 g, 0.79 m) in THF (1.2 L) at room temperature were successively added triethylamine (120 g, 1.19 mol) and methyl bromoacetate (158 g, 1.03 mol). The reaction mixture was stirred at RT for a further 12 h. The mixture was filtered and the residue was washed with excess THF. The combined filtrates were concentrated in vacuo and the residue was partitioned between EtOAc (2.5 L) and water. The organic phase was washed with brine, dried (Na₂SO₄) and concentrated in vacuo. The residue was chromatographed (SiO₂; 60-120 mesh, 4:1 hexane/EtOAc) to afford amine 4 (125 g; 49% yield) which was 98% pure by HPLC.

A purified sample was characterized:

Chiral HPLC (Daicel Chiralcel AD 4.6×250 mm column, 0.8% isopropanol/heptane isocratic, 30 min run) showed 100% e.e. (the retention time of the desired product is 7.00 min, while its enantiomer has a retention time of 5.20 min).

¹H NMR (400 MHz, CDCl₃) δ 7.12 (d, J=8.79 Hz, 2 H), 6.76 (d, J=8.35 Hz, 2 H), 3.70 (q, J=6.59 Hz, 1 H), 3.66 (s, 3 H), 3.17-3.29 (m, 2 H), 1.91 (br. s, 1 H), 1.32 (d, J=6.15 Hz, 3 H), 0.95 (s, 9 H), 0.17 (s, 6 H)

¹³C NMR (126 MHz, CDCl₃) δ 173.01, 154.61, 137.08, 127.61, 119.87, 57.02, 51.60, 48.55, 25.60, 24.06, 18.10, −4.49

[α]_{D}=−57.65° (c=8.4 mg/mL, MeOH, temperature=25.2° C.)

LRMS (M-glycine methyl ester+H)⁺: 235.3

### Synthesis of (S)-methyl N-((3-fluoro-4-methylphenoxy)carbonyl)-N-(1-(4-(tert-butyldimethylsilyloxy)phenyl)ethylamino)acetate (9)

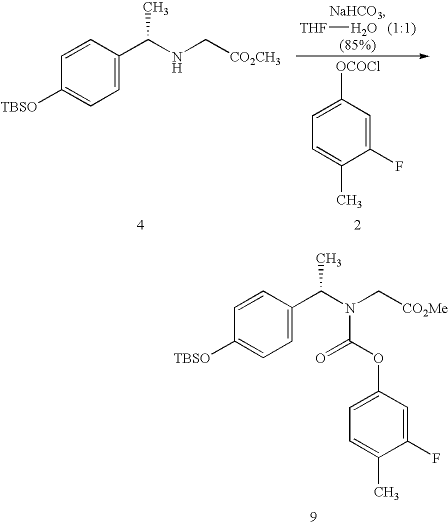

A 2 L 3 neck round bottom flask equipped with a mechanical stirrer was charged with amine 4 (120 g, 370 mmol), THF (600 mL) and saturated aqueous NaHCO₃ (500 mL). A solution of crude chloroformate 2 (86 g, 440 mmol) in THF (200 mL) was added dropwise over a period of 30 minutes to the mixture at 0° C. under an atmosphere of N₂. After the addition was complete, the mixture was allowed to warm up to room temperature and stirred at room temperature for an additional 30 min. Et₂O (600 mL) and H₂O (150 mL) were then added. The organic layer was washed with aqueous NaOH (1 N, 3×100 mL), H₂O (2×100 mL), and brine (2×100 mL). The organic layer was dried (Na₂SO₄) and concentrated in vacuo to afford the carbamate 9 (170 g, 97%) as a light yellow oil.

A purified sample was characterized:

¹H NMR (500 MHz, DMSO-d6) δ 7.22-7.34 (m, 3 H), 6.78-6.95 (m, 4 H), 5.33-5.44 (m, 1 H), 3.83-4.09 (m, 2 H), 3.59 (s, 3 H), 2.20 (s, 3 H), 1.57 and 1.49 (d, J=7.15 Hz 1:2 rotamers, 3 H), 0.94 (s, 9 H), 0.18 (s, 6 H)

¹³C NMR (126 MHz, DMSO-d6, 75° C.) δ 169.56, 160.08 (d, J=244.14 Hz, 1 C), 154.46, 153.39, 149.81 (d, J=10.19 Hz, 1 C), 132.82, 131.20, 128.37, 120.90 (d, J=17.84 Hz, 1 C), 119.47, 117.09, 108.68 (d, J=25.49 Hz, 1 C), 53.98, 51.51, 44.68, 25.39, 17.72, 16.71, 13.29, −4.70

¹⁹F NMR (376 MHz, DMSO-d6) δ −114.82 (s, 1 F)

[α]_{D}=−84.6° (c=12.4 mg/mL, MeOH, temperature=24.2° C.)

LRMS (M+Na)⁺: 498.3

HPLC Method: Gradient solvent system: from 50% A: 50% B to 0% A: 100% B(A=90% H₂O/10% MeOH+0.2% H₃PO₄; B=90% MeOH/10% H₂O+0.2% H₃PO₄) for 8 min; detection at 220 nm; Flow rate=2.5 mL/min; Zorbax SB C18 4.6×50 mm column; Retention time=9.39 min

### Synthesis of N-((3-fluoro-4-methylphenoxy)carbonyl)-N-(1-(4-hydroxyphenyl)ethyl amino)acetate (10)

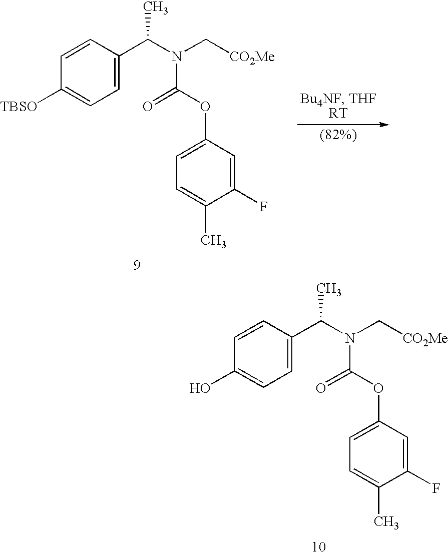

To a 1 L 3 neck round bottom flask equipped with a mechanical stirrer containing compound 9 (133 g, 0.28 mol) in THF (300 mL) at 0° C. was slowly added Bu₄NF (280 mL of a 1 M solution in THF; 280 mmol) over 30 minutes. After the addition was complete, the reaction mixture was allowed to warm up to room temperature and stirred for an additional 30 minutes. By the end of the addition, HPLC showed that the reaction was complete. Solvent was removed in vacuo, and the residue was partitioned between EtOAc (500 mL) and aqueous HCl (3×150 mL of a 1 N solution). The organic phase was washed with H₂O (150 mL) and brine (150 mL), then dried (MgSO₄) and concentrated in vacuo to afford the crude product, which was purified by flash column chromatography (25% to 40% EtOAc/Hexane) to afford the desired phenol 10 (91.98 g, 91% yield) as a light yellow oil.

A purified sample was characterized:

HPLC Method: Gradient solvent system: from 50% A: 50% B to 0% A: 100% B(A=90% H₂O/10% MeOH+0.2% H₃PO₄; B=90% MeOH/10% H₂O+0.2 % H₃PO₄) for 8 min; detection at 220 nm. Flow rate=2.5 ml/min. Zorbax SB C18 4.6×50 mm column; Retention time=4.43 min

¹H NMR (500 MHz, DMSO-d6) δ 9.43 (s, 1 H), 7.12-7.34 (m, 3 H), 6.70-7.01 (m, 4 H), 5.28-5.42 (m, 1 H), 3.78-3.93 (m, 2 H), 3.60 (s, 3 H), 2.21 (s, 3 H), 1.54, 1.47 (d, J=7.15 Hz, 1:2 rotamers, 3 H)

¹³C NMR (126 MHz, DMSO-d6, 75° C.) δ 169.56, 159.94 (d, J=244.14 Hz, 1 C), 156.60, 153.26, 149.70 (d, J=12.71 Hz, 1 C), 130.84, 130.07, 128.11, 120.78 (d, J=17.80 Hz, 1 C), 117.01, 114.97, 108.58 (d, J=25.43 Hz, 1 C), 53.82, 51.43, 44.43, 16.53, 12.61

¹⁹F NMR (376 MHz, DMSO-d6) δ −114.76 (s, 1 F)

[α]_{D}=−102.43° (c=12.2 mg/mL, MeOH, temperature=24.7° C.)

LRMS (M+Na)⁺: 384.1

### Synthesis of Glycine, N-[(3-fluoro-4-methylphenoxy)carbonyl]-N-[(1S)-1-[4-[2-(5-methyl-2-phenyl-4-oxazolyl)methoxy]phenyl]ethyl]methyl ester (11)

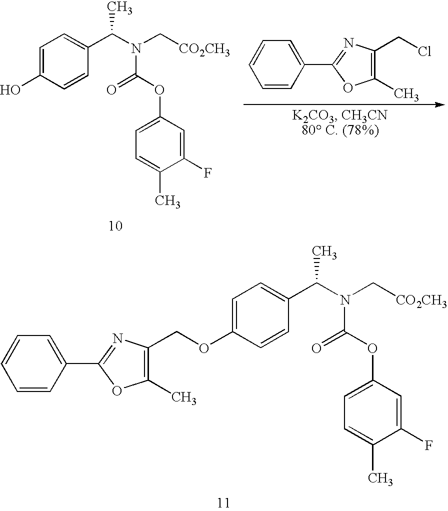

A 2 L 3 neck round bottom flask equipped with a mechanical stirrer was charged with compound 10 (100 g, 277 mmol), CH₃CN (500 mL), compound 3 (58.5 g, 282 mmol) and K₂CO₃ (76.5 g, 540 mmol). After the reaction mixture was stirred at 80° C. for 8 hours, it was cooled down to room temperature, then to 5° C. Saturated aqueous NH₄Cl (300 mL) was added via an additional funnel. The organic layer was separated and the aqueous layer was extracted with EtOAc (2×200 mL). The combined organic layers were washed with brine(150 mL), dried over anhydrous Na₂SO₄ and concentrated in vacuo. The crude product was purified by flash column chromatography (SiO₂; from 30% to 35% EtOAc/Hexane) to afford 11 (118 g, 80% yield) as a light yellow oil.

A purified sample was characterized:

¹H NMR (400 MHz, DMSO-d6, 80° C.) δ 7.93 (d, J=5.27 Hz, 2 H), 7.45-7.55 (m, 3 H), 7.33 (d, J=8.35 Hz, 2 H), 7.26 (t, J=8.57 Hz, 1 H), 7.04 (d, J=8.35 Hz, 2 H), 6.90 (d, J=10.55 Hz, 1 H), 6.83 (d, J=7.91 Hz, 1 H), 5.37 (q, J=6.74 Hz, 1 H), 3.95-4.07 (m, 1 H) 5.02 (s, 2 H), 3.84-3.94 (m, 1 H), 3.60 (s, 3 H), 2.43 (s, 3 H), 2.21 (s, 3 H), 1.54 (d, J=4.83 Hz, 3 H)

¹³C NMR (101 MHz, DMSO-d6, 80° C.) δ 169.4, 169.2, 159.8 (d, J=245.3 Hz, 1 C), 157.4, 153.1, 149.5 (d, J=10.1 Hz, 1 C), 146.9, 131.8, 131.0, 131.0, 129.9, 128.6, 128.0, 126.7, 125.3, 120.7 (d, J=18.2 Hz, 1 C), 116.9, 114.6, 108.4 (d, J=26.3 Hz, 1 C), 61.59, 53.9, 51.3, 44.7, 16.7, 13.1, 9.6

¹⁹F NMR (376 MHz, DMSO-d6, 80° C.): δ 115.3 (s, 1 F)

[α]_{D}=−98.3° (c=3.2 mg/mL, CH₂Cl₂, temperature=25.2° C.)

LRMS (M+H)⁺: 533.2
Synthesis of Compound Ia

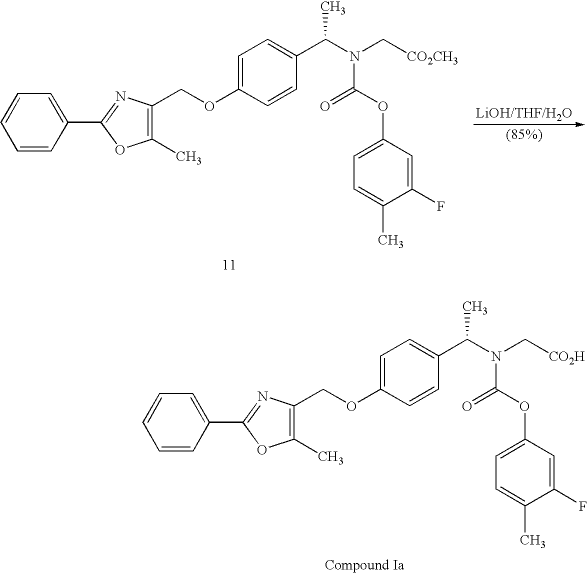

A 2 L 3-neck round bottom flask was charged ester 11 (115 g, 216 mmol), THF (800 mL), H₂O (400 mL) and LiOH.H₂O (22.67 g, 540 mmol). After the reaction mixture was stirred at room temperature overnight under N₂, the reaction was judged to be complete. by HPLC. The reaction mixture was diluted with EtOAc (200 mL), and the pH was adjusted with aqueous 1N HCl to pH=2. The organic layer was separated, and the aqueous layer was extracted with EtOAc (2×250 mL). The organic layer was washed with water (2×150 mL), dried (Na₂SO₄) and concentrated in vacuo to afford crude Compound Ia (110 g, 99% yield). HPLC analysis showed that the purity of this batch was 98.1%.

Recrystallization:

Crude Compound Ia (180 g) was dissolved in hot EtOH (700 mL) at 85° C. The mixture temperature was cooled to room temperature, then to 5° C. The slurry was filtered and the cake was washed with cold EtOH (2×100 mL). The white solid was dried under vacuum at 55° C. for 8 hours until a constant weight was obtained. The weight of the solid was 144 g (80% recovered yield). The chemical purity was determined by DAS to be 99.5% with 99.2% ee

¹H NMR (500 MHz, DMSO-d6; 24° C.) δ 12.71 (s, 1 H), 7.89-7.99 (m, 2 H), 7.45-7.59 (m, 3 H), 7.22-7.41 (m, 3 H), 6.99-7.10 (m, 2 H), 6.88-6.99 (m, 1 H), 6.78-6.88 (m, 1 H), 5.28-5.46 (m, 1 H), 4.99 (s, 2H), 3.80-4.03 (m, 1 H), 3.68-3.80 (m, 1 H), 2.44 (s, 3 H), 2.20 (s, 3 H), 1.56, 1.49 (d, J=7.15 Hz, rotamers, 3 H)

³C NMR (126 MHz, DMSO-d6; 24° C.) δ 171.2,170.7, 161.3, 159.4, 159.1, 157.8, 157.7, 154.0, 153.7, 150.2, 147.7, 133.4, 132.8, 132.2, 131.6, 130.6, 129.4, 128.7, 128.49, 127.1, 125.8, 121.4, 121.3, 117.8, 117.7, 114.8, 109.4, 109.2, 61.6, 54.6, 53.8, 45.5, 44.8, 18.0, 16.9, 13.9, 10.3 (note: extra peaks are due to rotamers as the spectrum was run at 24° C.)

¹⁹F NMR (471 MHz, DMSO-d6; 24° C.) δ −115.2 (s, 1 F)

HRMS(M+H)⁺=519.1945 (Δ=2.5 ppm);

[α]_{D}=−89.6° (c=9.100 mg/mL, CHCl₃, temperature=25° C.)

### Example 2

### Preparation of Compound Ia (Procedure 2)

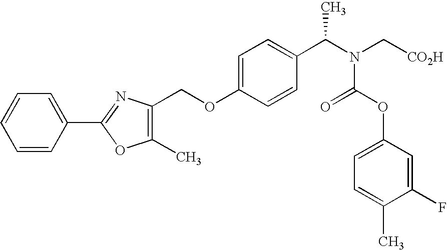 Preparation of Compound 13

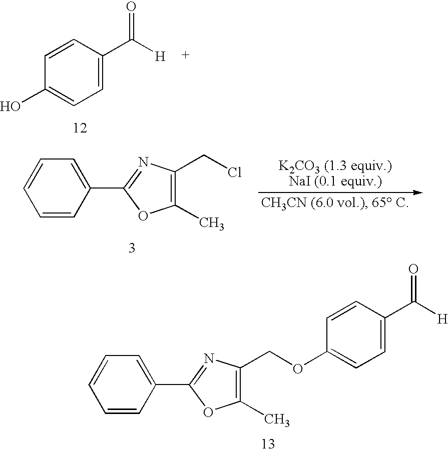

Into a 1000 mL 3-neck round bottom flask equipped with a mechanical stirrer, a thermal couple, a heating mantle, and a condenser was charged Compound 3 (30.2 grams, 145.4 mmol, 1.00 equiv.), Compound 12 (4-hydroxybenzaldehyde, 19.2 grams, 1.08 equiv.), potassium carbonate (26.3 grams, 1.31 equiv.), sodium iodide (2.40 grams, 0.11 equiv.), and 181.0 mL CH₃CN (6.0 vol./g input of Compound 3). The resulting slurry was stirred at ambient temperature for 15-30 min. The slurry was then heated to 65.5° C. and continually stirred at 65.5° C. for ˜2.0 h. Thereafter, the batch was cooled to 2-10° C. Then 420 mL (˜14.0 vol./g input of Compound 3) of water was added into the reaction mixture in <1 min. An off-white slurry was formed uniformly. The slurry was heated back to ˜55-60° C. and held at that temperature for ˜1 h. The batch was cooled to ambient temp. in 2-3 h. and continuously stirred at ambient temperature for 18 h. The slurry was filtered through a Buchner funnel with a #1 Whatman® filter paper to collect the crystalline solid. The wet cake was washed with water (90 mL×3). The wet cake was de-liquored with vacuum for 30 min at ambient temp. The estimated weight of the product was ˜42.6 (g) with an isolated yield of 96.8%. The product (Compound 13) can be further purified by re-crystallization from acetone. mp 117.2° C. ¹H NMR (CDCl₃): δ 2.46 (s, 3H), 5.08 (s, 2H), 7.13-7.15 (m, 2H), 7.44-7.46 (m, 3H), 7.85-7.87 (m, 2H), 8.01-8.04 (m, 2H), 9.90 (s, 1H). ¹³C NMR (CDCl₃): δ 10.4, 62.4, 115.0, 126.1, 127.2, 128.7, 130.1, 130.2, 131.2, 131.9, 147.4, 160.1, 163.4, 190.7. IR (KBr): 2919.4, 2794.3, 2725.2, 1691.8, 1643.8, 1600.4, 1575.2, 1556.1, 1508.0, 1484.4, 1302.1, 1245.3, 1212.3, 1159.2, 998.2, 869.6, 776.3, 715.0, 698.8, 692.6 cm⁻¹. HRMS calcd for C₁₈H₁₆NO₃ [M+H]⁺: 294.1130, found, 294.1131. Anal. Calcd for C₁₈H₁₅NO₃: C, 73.70; H, 5.15; N, 4.77. Found: C, 73.49; H, 4.97; N, 4.60.
Preparation of Compound 15

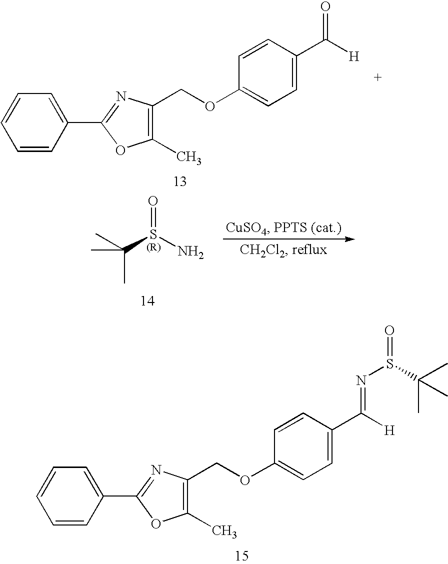

To a 1000 mL jacketed reactor equipped with a temperature probe, a mechanic agitator, and a condenser, and a bath circulator was charged Compound 13 (50.0 g, 170.5 mmol, 1.0 equiv.), Compound 14 (25.0 g, 206.3 mmol, 1.21 equiv.), copper sulfate (CuSO₄) (81.6 g, 511.4 mmol, 3.0 equiv.), pyridinium p-toluenesulfonate (PPTS) (2.14 g, 8.5 mmol, 0.05 equiv.) and 300 mL methylene chloride. The resulting slurry was heated to reflux and was held at reflux for ˜4-6 hours. The reaction mixture was then allowed to cool down to ambient temperature. The insoluble inorganic salts were then filtered off. The inorganic salt cake was washed with 100 mL×2 of methylene chloride. The filtrate and washes were combined as a methylene chloride solution. The organic solution was washed with aqueous 5% wt ammonium acetate (aq, 250 mL×2) and water (250 mL). After the phases were separated, the organic layer was concentrated to ˜175-200 mL via distillation under atmospheric pressure. Heptane (1200 mL) was charged to the concentrated methylene chloride solution in 1.5 hours while maintaining the batch temperature at ˜33° C. After charging heptane, the resulting slurry was allowed to cool down to ˜5° C. in >1 hr and stirred at ˜5° C. for 20 min. The solids were then collected by filtration, followed by washing with 160 mL of heptane. The wet cake was dried at 45° C. under reduced pressure to give ˜56 g of Compound 15 as a bright yellow solid. The isolated yield is 85.8%. The product can be further purified by re-crystallization from methylene chloride and heptane. mp 160.0° C. ¹H NMR (CDCl₃): δ 1.09 (s, 9H), 2.29 (s, 3H), 4.9 (s, 2H), 6.92-6.94 (m, 2H), 7.27-7.30 (m, 3H), 7.64-7.66 (m, 2H), 7.84-7.87 (m, 2H), 8.35 (s, 1H). ¹³C NMR (CDCl₃): δ 10.2, 22.2, 57.2, 62.0, 114.8, 125.8, 127.0, 127.2, 128.4, 129.9, 130.9, 131.2, 147.0, 159.7, 161.3, 161.6. IR (KBr): 2972.6, 2922.4, 2868.5, 1639.8. 1597.8, 1563.7, 1508.4, 1467.8, 1401.2, 1302.3, 1248.4, 1240.6, 1168.7, 1076.3, 987.1, 876.9, 837.2, 775.2, 716.2, 698.0, 650.4, 593.0, 521.9 cm⁻¹. Anal. Calcd for C₂₂H₂₄N₂O₃S: C, 66.64; H, 6.10; N, 7.06. Found: C, 66.51; H, 5.88; N, 6.99. [α]_{D}=+0.5890 (c 0.89, MeOH).
Preparation of Compound 16

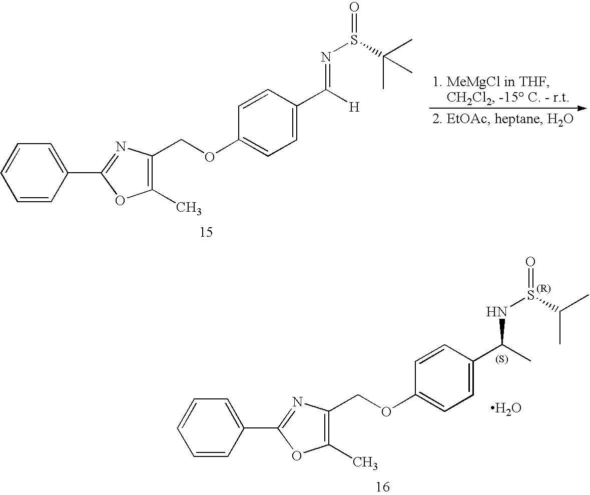

To a 500 mL 3-neck jacketed flask equipped with a temperature probe, a mechanic agitator, and a condenser, and a bath circulator was charged Compound 15 (15 g, 37.8 mmol, 1.0 equiv.), and 225 mL (15 vol. of each g of Compound 15 input) of methylene chloride to form a light yellow solution at ambient temp. The resulting solution was cooled to ˜−15° C. Methyl magnesium chloride (3 N in THF, 32.0 mL, 2.54 equiv.) was charged to the solution over 20 min while maintaining the batch temperature below −7° C. The reaction was stirred at ˜−10° C. for 5 h. Then the reaction mixture was allowed to warm up to ˜0° C. and held for 30 min at 0° C. 5.6 mL of acetone was charged to the reaction mixture over 20 min while maintaining the batch temperature <20° C. The resulting solution was stirred at room temperature for 30 min. 72 mL of aqueous 10% wt. NH₄OAc was charged to the reaction mixture. Subsequently, the pH of the reaction mixture was adjusted to 4.0-5.0 (top aq layer) with 1 N HCl (aq) solution in 15 min. The bi-phasic system was stirred for ˜20 min at ambient temp. After the phases were separated, the bottom organic layer was retained followed by washing with aqueous 5% wt. NaHCO₃ (60 mL) and water (60 mL).

The isolated organic solution was concentrated to 45-60 mL volume through distillation at atmospheric pressure. 150 mL of ethyl acetate was charged to the concentrated solution at ambient temp. The solution was distilled to concentrate to 75 mL through vacuum distillation while maintaining the batch temp. at 50-55° C. Another 150 mL ethyl acetate was charged to the residual solution followed by vacuum distillation to reduce the volume to ˜75 mL. When necessary, the ethyl acetate charge-distillation sequence was repeated until % (v/v) contents of methylene chloride and THF were both <1% relative to EtOAc by GC analysis. The solution was cooled down to ˜40° C. followed by adding 1.5 mL of water. 45 mL of heptane was added while maintaining the batch temp at 35-40° C. The resulting solution was seeded with 225 mg of Compound 16 seed crystals, then was stirred at 35-40° C. for 30 min. To the resulting white slurry, 225 mL of heptane was added over a period of 1 h while maintaining the batch temp at 35-40° C. The slurry was cooled to ambient temp over 1 h, and stirring was continued at ambient temp for 14 h. The solids were collected through filtration. The product was washed with heptane (80 mL×2). The wet product cake was de-liquored for 1 h at ambient temp. followed by drying at ambient temp under vacuum for >12 h. 15.2 g of Compound 16 was obtained as a white solid, with an isolated yield of 88%. The product can be further purified by re-crystallized from EtOAc, heptane, and water. mp 124.0° C. ¹H NMR (CDCl₃): δ 1.17 (s, 9H), 1.50 (d, J=6.5 Hz, 3H), 2.40 (s, 3H), 2.53 (m, 1H), 3.40 (s, 1H), 4.52 (m, 1H), 4.96 (s, 3H), 6.97 (d, J=8.3 Hz, 2H), 7.25 (d, J=8.6 Hz, 2H), 7.40 (m, 3H), 7.99-8.00 (m, 2H). ¹³C NMR (CDCl₃): δ 10.3, 22.4, 24.8, 53.9, 55.3, 62.1, 114.6, 125.9, 127.2, 127.9, 128.5, 129.9, 131.8, 135.7, 146.9, 157.7, 159.7. IR (KBr): 3391.5, 3150.6, 2975.6, 2930.9, 1695.6, 1643.9, 1610.0, 1583.6, 1557.0, 1512.2, 1492.6, 1460.2, 1448.2, 1366.0, 1231.5, 1180.9, 1069.7, 1024.9, 1002.8, 949.0, 867.0, 832.2, 775.1, 713.9, 690.8, 647.1, 551.2 cm⁻¹. Anal. Calcd for C₂₃H₃₀N₂O₄S: C, 64.16; H, 7.02; N, 6.50; S, 7.44. Found: C, 64.22; H, 7.19; N, 6.41, S, 7.33. [α]_{D}=−60.68° (c 2.03, MeOH).
Preparation of Compound 17

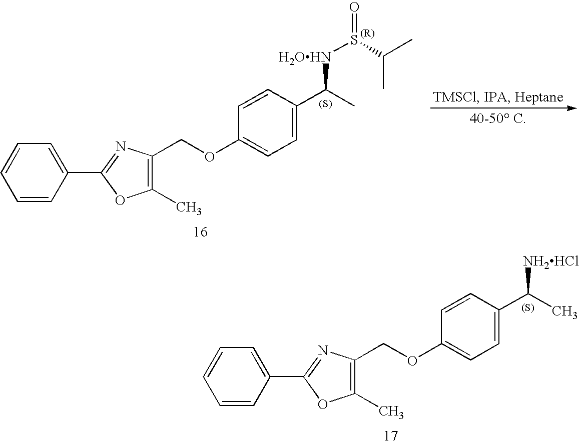

Into a 1000 mL jacketed reactor equipped with a overhead stirrer, a thermal couple, a condenser, a heating mantle, and a bath circulator was charged Compound 16 (40.0 grams, 92.9 mmol, 1.0 equiv.), isopropanol (IPA, 280 ml, 7.0 ml per g input of Compound 16). The resulting slurry was stirred at ambient temperature for 5-10 min. The slurry was then heated to 40° C. to achieve full dissolution. It was then distilled under vacuum at ˜40° C. until the KF of the IPA solution was <0.1% wt. while maintaining the volume of the solution by adding fresh isopropanol. TMSCl (18.1 mL, 1.5 equiv.) was charged slowly into the solution at ˜40° C. for ˜1-1.5 h. White solids precipitated out to form a slurry. The slurry was stirred at ˜40° C. for 30 min, after which 600 mL of n-heptane was added at ˜40° C. in 3 h followed by holding at ˜40° C. for ˜1 h. The white slurry was cooled to ambient temp (˜20° C.) and held at ambient temperature for >6 h. The slurry was filtered through a Buchner funnel with a #1 Whatman® filter paper to collect the crystalline solid. The wet product cake was washed with a mixture of isopropanol (40 mL) and n-heptane (160 mL), followed by additional n-heptane (200 mL×3). The wet cake was de-liquored under nitrogen under vacuum for 30 min at ambient temp. The product was dried at 50° C. in a vacuum oven overnight to give 29.2 g of Compound 17 as a white solid. Yield=91.7%. The product can be further purified by re-crystallization from EtOH and MTBE (methyl t-butyl ether). mp 200.0° C. ¹H NMR (CD₃OD): δ 1.61 (d, J=6.8 Hz, 3H), 2.45 (s, 3H), 4.41 (m, 1H), 5.03 (s, 2H), 7.10-7.13 (m, 2H), 7.40-7.42 (m, 2H), 7.47-7.51 (m, 3H), 7.96-8.00 (m, 2H). ¹³C NMR (CD₃OD): δ 10.7, 21.0, 52.3, 63.3, 117.0, 127.5, 128.6, 129.7, 130.5, 132.2, 132.5, 133.4, 149.5, 160.8, 161.9. IR (KBr): 3257.5, 2945.1, 2899.0, 2806.8, 1603.4, 1506.1, 1465.1, 1219.3, 1147.7, 999.1, 819.9 cm⁻¹. Anal. Calcd for C₁₉H₂₁ClN₂O₂: C, 66.17; H, 6.13; N, 8.12; Cl, 10.28. Found: C, 65.88; H, 5.91; N, 8.04, Cl, 10.48. [α]_{D}=−60.680 (c 2.03, MeOH).
Preparation of Compound 18

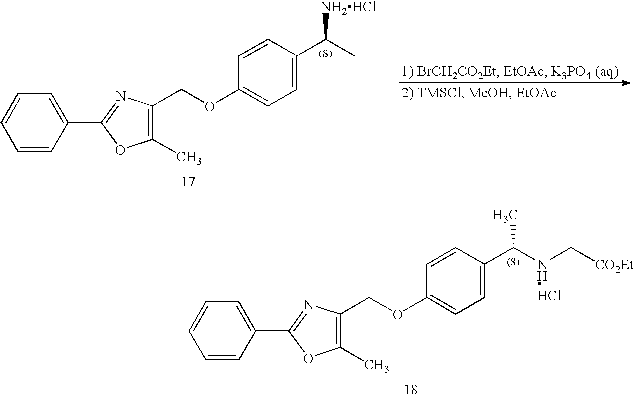

Compound 17 (25.0 g, 72.6 mmol, 1.0 equiv.) was charged to a round bottom flask, followed by EtOAc (10 mL/g) and water (4 mL/g). At ambient temperature, the mixture was allowed to agitate for 15 minutes until full dissolution was reached. 20 wt % aqueous K₃PO₄ (124.0 g) was then charged in one portion, bringing the pH to ˜10. Subsequently, ethyl bromoacetate (12.7 g, 76.0 mmol,1.05 equiv.) was charged in one portion. The biphasic reaction mixture was agitated at ambient temperature for 1.5-2 hours. 20 wt % aqueous K₃PO₄ (30.0 g) was then charged into the reaction. The reaction mixture was then heated to 40-45° C. and held at 40-45° C. for 5-7 h. If the reaction was not complete by HPLC (<4 AP of Compound 17) monitoring at this point, the reaction mixture was cooled to ambient temperature and held overnight with agitation. When the reaction was complete, the agitation was stopped and the phases were allowed to settle at ambient temperature. The bottom aqueous layer was then removed. The upper organic layer was washed first with phosphate buffer (250 mL, pH 5-5.5), followed by water (250 mL). From the retained top organic layer, water was removed by azeotropic distillation under vacuum to <1500 ppm, and EtOAc was added to bring the volume to the original volume after distillation. Next, MeOH (4.64 g, 145.0 mmol) was added to the EtOAc solution and the mixture was heated to 45° C. TMSCl (9.45 g, 87.0 mmol, 1.2 equiv.) was slowly added over 45-60 minutes; a slurry formed when 15-40% of the TMSCl had been added. After addition was complete, the slurry was allowed to stir at 45-50° C. for 1 h, then cooled to ambient temperature over 1 h, and allowed to stir for an additional 1-2 h before filtration and EtOAc wash (75 mL). The wet cake was dried under vacuum at ambient temperature for 72 h to give Compound 18 as a white powder (26.4 g). Yield=84.6%. The product can be further purified by re-slurrying in EtOA followed by filtration. mp 182.3° C. ¹H NMR (400 MHz, CDCl₃): δ 1.19 (t, J=7.14 Hz, 3H), 1.91 (d, J=6.81 Hz, 3H), 2.39 (s, 3H), 3.41 (d, J=16.92 Hz, 1H), 3.64 (d, J=16.92 Hz, 1H), 4.14 (dd, J=7.18 Hz, 2H), 4.58 (m, 1H), 4.94 (s, 2H), 7.02 (d, J=8.79 Hz, 2H), 7.37-7.41 (m, 3H), 7.55 (d, J=8.79 Hz, 2H), 7.97 (dd, J=4.61, 3.30 Hz, 2H), 10.18 (s, 1H), 10.39 (s, 1H). ¹³C NMR (CDCl₃, 100 MHz): δ 10.4, 13.9, 20.0, 44.6, 58.1, 62.1, 115.5, 126.0, 127.3, 127.5, 128.6, 129.7, 130.0, 131.6, 147.1, 159.3, 160.0, 165.7. IR (KBr): 3416.8, 2934.3, 2711.6, 2610.9, 2462.4, 1757.3, 1630.1, 1614.1, 1584.4, 1554.5, 1485.7, 1430.0, 1449.2, 1412.9, 1395.4, 1329.5, 1309.9, 1269.9, 1024.4, 979.2, 713.9, 705.8, 693.0, 687.2 cm⁻¹. Anal. Calcd for C₂₃H₂₇ClN₂O₄: C, 64.01; H, 6.31; N, 6.50; Cl, 8.22. Found: C, 64.00; H, 6.36; N, 6.44; Cl, 8.26. [α]_{D}=−27.56° (c 1.04, MeOH).

In the first step (above), another base, triethylamine, can be used in place of K₃PO₄ under anhydrous conditions.
Preparation of Compound Ia

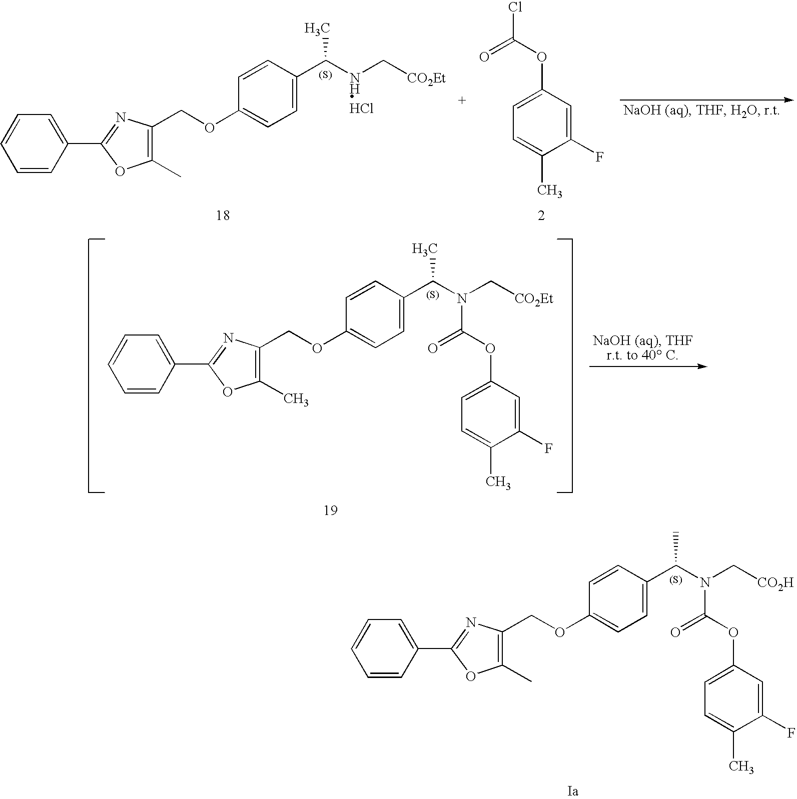

To a 250 mL glass reactor equipped with an agitator, a temperature probe and a condenser, was added 25.0 g of Compound 18 (58.01 mmol), 150 mL THF, and 10.0 mL water. After the reaction mixture was stirred to obtain a homogenous solution and cooled to 0-5° C., 10 NNaOH (aq., 14.5 mL, 145 mmol, 2.5 equiv.) was added over ˜10-15 minutes. Chloroformate 2 (11.5 g, 60.98 mmole, 1.05 equiv.) was then added in ˜20 minutes while maintaining the batch temperature at ≦5° C. After the addition was complete, the reaction mixture was allowed to warm up to ambient temperature and the reaction was monitored by HPLC for completion. After the acylation reaction was complete, additional 10.0 NNaOH (aq., ˜40.6 mL, 406 mmol, 7.0 equiv.) was added within 5-10 minutes. The reaction mixture was stirred at ˜35° C. for ˜4-6 h before cooling to ambient temperature and was held overnight at ambient temperature. HPLC indicated that the reaction was completed (Compound 19). The bottom aqueous layer was removed upon phase separation. About 375.0 mL deionized water was then added to the organic layer to form a solution. The pH of the resulting solution was adjusted to ˜6.5-7.5 using 1 NHCl (aq). Seed crystals (˜0.25 g) of Compound Ia were added. The pH adjustment was continued slowly over 2-4 h using 1 N HCl (aq) to the final pH of 2.2. The slurry was stirred at ambient temperature overnight. The solid was filtered via a Buchner funnel and was washed with 125 mL×4 water. The wet cake was dried in a vacuum oven at ˜40-50° C. under house vacuum for 22 h to provide 28.35 g of Compound Ia. Yield: 92.8%. The product can be further crystallized from EtOH to improve the purity and enantiomeric purity to 100.0%. mp 152.6° C. ¹H NMR (DSMO-d₆, 95.0° C.): δ 1.55 (d, J=6.37 Hz, 3 H), 2.21 (s, 3H), 2.43 (s, 3 H), 3.77 (br d, J=17.8 Hz, 1H), 3.95 (br d, J=18.0 Hz, 1H), 5.02 (s, 2H), 5.37 (q, J=6.96 Hz, 1H), 6.85-6.92 (m, 2H), 7.04 (d, J=8.35 Hz, 2H), 7.25 (t, J=8.57 Hz, 1H), 7.34 (d, J=8.35 Hz, 2H), 7.47-7.52 (m, 3H), 7.94 (d, J=7.69 Hz, 2H). ¹³C NMR (DMSO-d₆, 22.8° C.): δ 9.99, 13.64, 16.69, 17.71, 44.52, 45.29, 53.58, 54.39, 61.37, 108.91, 109.15, 114.58, 117.42, 117.54, 120.99, 121.15, 125.59, 126.82, 128.25, 128.50, 129.10, 130.35, 131.36, 131.42, 131.93, 132.59, 133.16, 147.48, 149.74, 149.84, 149.96, 153.45, 153.79, 157.48, 157.56, 158.83, 161.30, 170.43, 170.96. Extra peaks in ¹³C NMR spectra are due to the presence of rotamers. Anal. Calcd for C₂₉H₂₇FN₂O₆: C, 62.17; H, 5.24; N, 5.40; F, 3.66. Found: C, 67.18; H, 5.25; N, 5.34; F, 3.86. [α]_{D}=−92.80° (c 0.912, CHCl₃).

### Example 3

### Preparation of Compound Ib

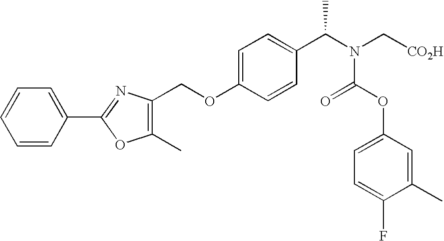

### Synthesis of 4-fluoro-3-methylphenyl carbonochloridate (21)

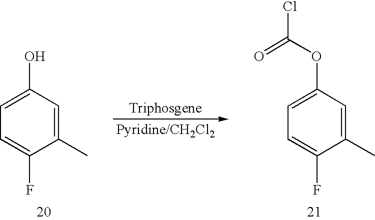

To a 0° C. solution of 4-fluoro-3-methylphenol 20 (10 g, 89.2 mmol) and triphosgene (8.7 g, 29.4 mmol) in CH₂Cl₂ (80 mL) was added pyridine (7.2 mL, 89.2 mmol) dropwise over 10 min. The reaction mixture was allowed to warm to RT, then was heated to 45° C. and stirred at 45° C. for an additional 2 h. The reaction was then cooled to RT, and volatiles were removed in vacuo. Anhydrous Et₂O (300 mL) was added to the residue, and the resulting slurry was filtered. The filtrate was concentrated in vacuo to afford crude 4-fluoro-3-methylphenyl chloroformate (17 g, >100% yield) as an oil, which was immediately used in the next step without further purification.

### Synthesis of (S)-methyl 2-((1-(4-(tert-butyldimethylsilyloxy)phenyl)ethyl)((4-fluoro-3-methylphenoxy)carbonyl)amino)acetate (22)

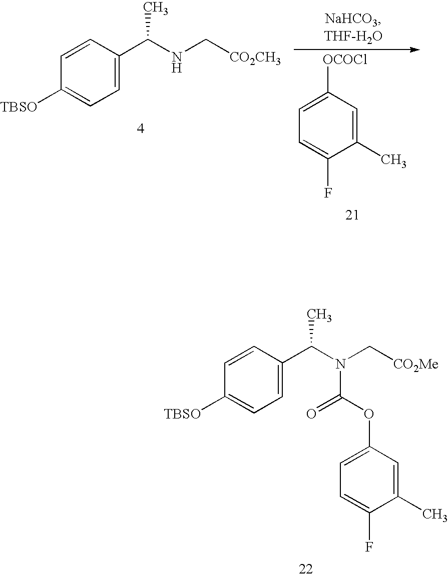

To a 5° C. mixture of amine 4 (24.0 g, 248 mmol), THF (120 mL), and saturated aqueous NaHCO₃ (100 mL) in a 500 mL round bottom flask (equipped with a mechanical stirrer) was added dropwise over 10 min a solution of crude chloroformate 21 (16.8 g, 89.2 mmol) in THF (10 mL) under an atmosphere of N₂. After the addition was complete, the mixture was allowed to warm to RT. The organic layer was separated, and the aqueous layer was extracted with EtOAc (200 mL). The combined organic extracts were concentrated in vacuo, and the residue was taken up in hexanes and washed with 1N aqueous NaOH (2×40 mL) and H₂O (2×40 mL). The organic layer was dried (Na₂SO₄), filtered, and concentrated in vacuo to afford the desired carbamate product 22 (35 g, 99% yield) as an oil.

### Synthesis of (S)-methyl 2-(((4-fluoro-3-methylphenoxy)carbonyl)(1-(4-hydroxyphenyl)ethyl)amino)acetate (23)

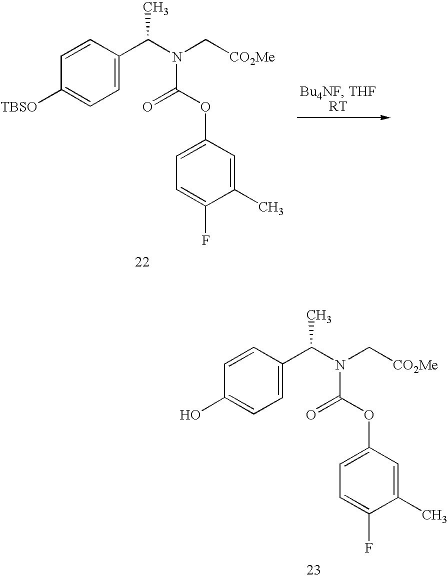

Multiple lots of compound 22 were synthesized, combined and used in the next reaction as follows. To a 10° C. solution of compound 22 (135 g, 0.28 mol) in THF (300 mL) was slowly added Bu₄NF (280 mL of a 1 M solution in THF; 280 mmol) over 30 min. After addition was complete, the reaction mixture was allowed to warm up to RT and stirred at RT for an additional 30 min. Volatiles were removed in vacuo, and the residue was taken up in hexanes and washed with H₂O (2×400 mL). The organic phase was dried (Na₂SO₄) and concentrated in vacuo. The residue was triturated with Et₂O to afford the product phenol 23 (100 g, 98% yield) as a white solid.

### Synthesis of Glycine, N-[(4-fluoro-3-methylphenoxy)carbonyl]-N-[(1S)-1-[4-[2-(5-methyl-2-phenyl-4-oxazolyl)methoxy]phenyl]ethyl]methyl ester (24)

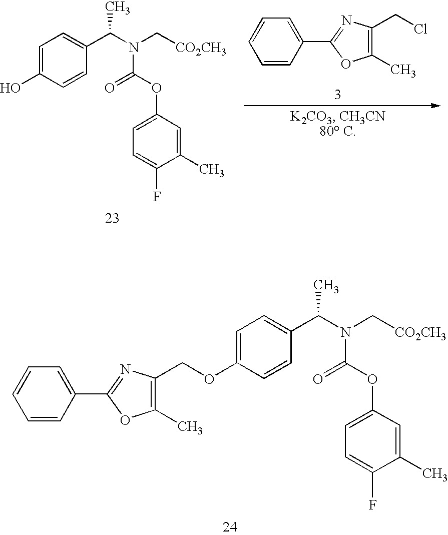

A mixture of phenol 23 (100 g, 277 mmol), chloride 3 (57.4 g, 277 mmol), and K₂CO₃ (76 g, 554 mmol) in CH₃CN (500 mL) was stirred at 85° C. for 3 h, then was cooled to RT. The reaction was diluted with toluene (200 mL) and filtered through a pad of Celite®. The filtrate was concentrated in vacuo to afford the crude product, which was chromatographed (SiO₂; 20% EtOAc/Hexane) to afford compound 24 (138 g, 93% yield) as a light yellow oil.
Synthesis of Compound Ib

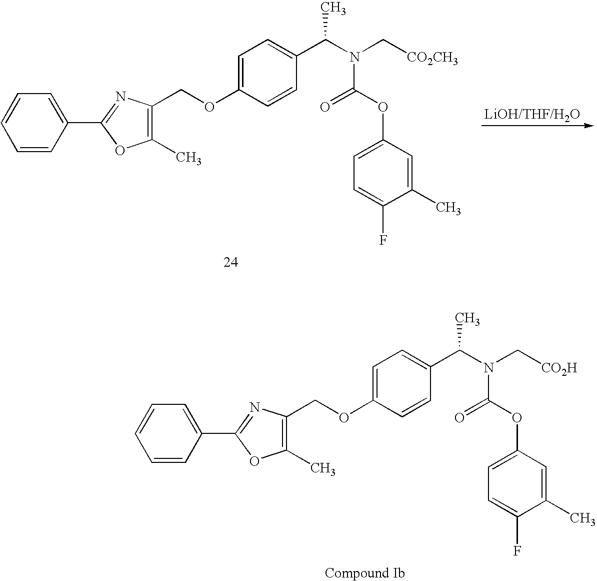

A mixture of ester 24 (135 g, 253 mmol), THF (800 mL), H₂O (400 mL) and LiOH.H₂O (26.5 g, 632 mmol) was stirred at RT overnight and then diluted with EtOAc (500 mL). The pH was adjusted with aqueous 1N HCl to ˜2. The organic layer was separated, dried (Na₂SO₄), and concentrated in vacuo to afford crude Compound Ib (125 g, 95% yield). HPLC analysis showed that the purity of this batch was 98.1%.

Recrystallization:

Crude Compound Ib (240 g, from multiple batches) was dissolved in hot EtOH (3 L) at 85° C. The mixture was cooled to RT, then to 5° C. The slurry was filtered, and the filter cake was washed with cold EtOH (2×100 mL). The white solid was dried in vacuo at 55° C. for 6 h. The weight of the solid was 192 g (80% recovered yield). The chemical purity was determined to be 98.6%. This batch of partially purified Compound Ib (192 g) was dissolved in hot EtOH (2 L) and hot MeOH (500 mL). EtOAc (500 mL) was added dropwise until a precipitate was formed. The mixture was cooled to RT, then to 0° C. for 30 min. The solid was collected by filtration and was dried in vacuo at 60° C. for 5 h until a constant weight was obtained. The weight of the solid was 188 g (98% recovered yield). The chemical purity was determined to be 99.6%, with >99.9% ee.

1H NMR (500 MHz, DMSO-d6; 24° C.) δ 12.70 (br. s., 1 H), 7.89-8.00 (m, 2 H), 7.45-7.57 (m, 3 H), 7.28-7.41 (m, 2 H), 7.10-7.20 (m, 1 H), 6.98-7.08 (m, 3 H), 6,87-6.96 (m, 1 H), 5.31-5.47 (m, 1 H), 5.01 (s, 2 H), 3.67-4.03 (m, 2 H), 2.46 (s, 3 H), 2.22 (s, 3 H), 1.43-1.63 (m, 3 H).

¹³C NMR (126 MHz, DMSO-d6; 24° C.) δ 171.0, 170.5, 158.8, 158.7, 157.5, 156.8, 154.0, 153.8, 147.5, 146.8, 133.3, 132.7, 131.9, 130.4, 129.1, 128.4, 128.2, 126.8, 125.6, 125.2, 125.0, 124.4, 120.6, 115.4, 115.2, 114.6, 61.3, 54.3, 53.5, 45.3, 44.5, 17.7, 16.7, 14.0, 10.0 (note: extra peaks are due to rotamers as the spectrum was run at 24° C.).

¹⁹F NMR (471 MHz, DMSO-d6; 24° C.) δ−122.0 (s, 1 F).

HRMS (M+H)⁺=519

[α]_{D}=87.44° (CHCl₃, temperature=25° C., 589 nm).

Theoretical elemental analysis calculation for C₂₉H₂₇FN₂O₆: C, 67.17%; H, 5.24%; N, 5.40%; F, 3.66%. Average values found from two separate elemental analysis tests: (C₂₉H₂₇FN₂O₆): C, 67.08%; H, 5.49%; N, 5.33%; F, 3.57%.

### Example 4

### Preparation of Compound Ic

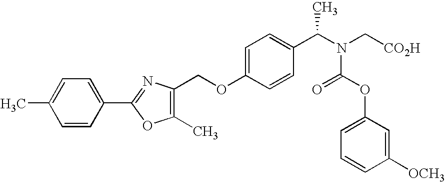

### Synthesis of 3-methoxyphenyl carbonochloridate (26)

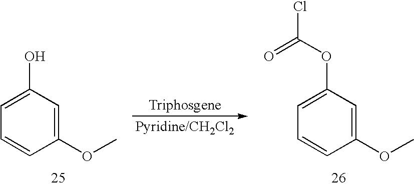

To a 0° C. solution of 3-methoxyphenol 25 (60 g, 483 mmol) and triphosgene (48 g, 161 mmol) in CH₂Cl₂ (500 mL) was added pyridine (39.1 mL, 483 mmol) dropwise over 1 h. The reaction mixture was then heated at 50° C. for an additional 1 h, then was cooled to RT, and volatiles were removed in vacuo. Anhydrous Et₂O (200 mL) was added to the residue, and the resulting slurry was filtered. The filtrate was concentrated in vacuo to afford crude 3-methoxyphenyl carbonochloridate 26 (89 g, 98.7% yield) as an oil, which was immediately used in the next step without further purification.

### Synthesis of (S)-methyl 2-((1-(4-(tert-butyldimethylsilyloxy)phenyl)ethyl)((3-methoxyphenoxy)carbonyl)amino)acetate (27)

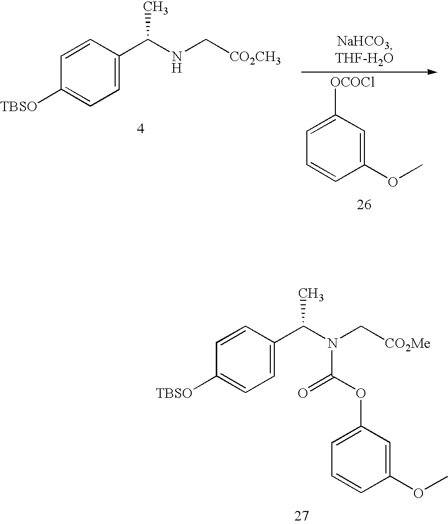

To a RT solution of amine 4 (60 g, 186 mmol) in THF (200 mL), saturated aqueous NaHCO₃ (200 mL), and H₂O (100 ml) was added the crude chloroformate 26 (26 g, 139 mmol) dropwise over 30 min. After the addition was complete, the mixture was stirred at RT for an additional 30 min, then was diluted with Et₂O (200 mL). The organic phase was washed with H₂O (200 mL), 1N aqueous NaOH (7×150 mL), 1N aqueous HCl (200 mL), H₂O (2×200 mL), then dried (Na₂SO₄) and concentrated in vacuo to provide the crude carbamate 27 (80 g, >100%) as a yellow oil.

### Synthesis of (S)-methyl 2-((1-(4-hydroxyphenyl)ethyl)((3-methoxyphenoxy)carbonyl)amino)acetate (28)

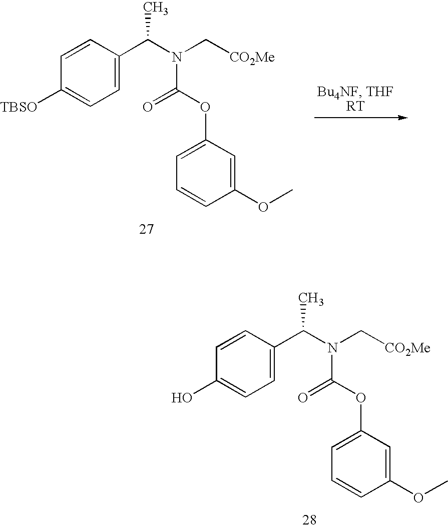

To a 0° C. solution of carbamate 27 (80 g, 222 mmol) in THF (200 mL) was slowly added Bu₄NF (200 mL of a 1 N solution in THF; 200 mmol) over 30 min. After the addition was complete, the reaction mixture was stirred at 0° C. for an additional 15 min. Volatiles were removed in vacuo, and the residue was taken up in Et₂O (300 mL). The organic phase was washed with H₂O (200 mL), 1N aqueous HCl (4×100 mL), and H₂O (2×100 mL), then was dried (Na₂SO₄), and concentrated in vacuo to provide the crude phenol 28 as a yellow oil. This material was chromatographed (SiO₂, 330 g ISCO column, continuous gradient from 0-50% EtOAc in hexane over 50 min, held at 1:1 EtOAc:hexane for 30 min) to afford phenol 28 (62 g, 92.8% yield) as a light yellow oil.

### Synthesis of 4-(chloromethyl)-5-methyl-2-p-tolyloxazole (29)

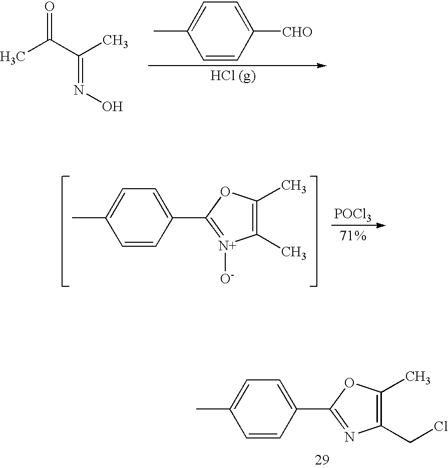

To a solution of p-tolualdehyde(100.0 g, 0.832 mol) in EtOAc (400 mL) was added 2,3-butanedione monoxime (80.0 g, 0.791 mol). The resulting solution was cooled to 0° C. and HCl (g) was bubbled through the solution for 10 min. The reaction mixture was stirred overnight at RT, then was concentrated to half the original volume and cooled to 0° C. The solids were filtered off and rinsed with cold EtOAc (100 mL) to afford a white solid. This crude N-oxide was dissolved in CHCl₃ (500 mL), and POCl₃ (81.0 mL, 0.870 mol) was added at RT in one portion. The reaction mixture was heated to 60° C. with stirring for 24 h, then was cooled to RT and concentrated in vacuo. The residue was taken up in EtOAc (750 mL) and cooled to 0° C.; H₂O (500 mL) was added, and the pH of the mixture was carefully adjusted to >8 by portionwise addition of solid NaHCO₃. The organic phase was washed with brine (100 mL), dried (MgSO₄) and concentrated in vacuo. The crude product was dissolved in EtOAc (700 mL) and filtered through a 2 inch plug of silica gel, which was rinsed with EtOAc (75 mL). The filtrate was concentrated in vacuo to give provide the oxazole chloride 29 (107 g, 61%) as a white solid.

M.P. 91-92° C.

¹H NMR (400 MHz, CDCl₃) δ 2.39 (s, 3H, CH₃), 2.41 (s, 3H, CH₃), 4.55 (s, 2H, CH₂), 7.24 (d, 2H, Ar—H), 7.88 (d, 2H, Ar—H)

¹³C NMR (400 MHz, CDCl₃) δ 10.29, 21.43, 37.32, 124.50, 126.12, 129.37, 132.69, 140.46, 146.15, 160.23

LC/MS m/e 221 (M+)

Calcd for C₁₂H₁₂ClNO: C, 65.01; H, 5.45; N, 6.31; Cl, 15.99. Found: C, 64.96; H, 5.71; N, 6.19; Cl, 16.04.

### Synthesis of Glycine, N-[(4-fluoro-3-methylphenoxy)carbonyl]-N-[(1S)-1-[4-[2-(5-methyl-2-phenyl-4-oxazolyl)methoxy]phenyl]ethyl]methyl ester (30)

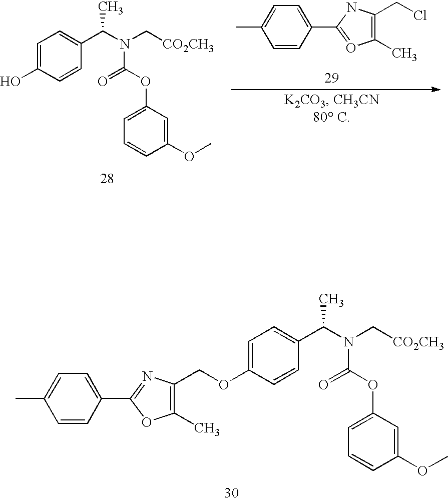

To a solution of phenol 28 (62 g, 172 mmol) in CH₃CN (800 mL), were successively added K₂CO₃ (124 g, 900 mmol) and chloride 29 (42 g, 189 mmol). The reaction mixture was stirred at reflux (oil bath temperature=94° C.) for 5.5 h, then was cooled to RT and filtered. The solids were thoroughly washed with EtOAc (3×50 mL). The combined filtrates were concentrated in vacuo. The residue was chromatographed (SiO₂, 330 g ISCO column (×2), continuous gradient from 0-35% EtOAc in hexanes over 50 min, then held at 35% EtOAc in hexane for 30 min) to provide the phenol ether 30 (90 g, 96%) as a colorless oil.
Synthesis of Compound Ic

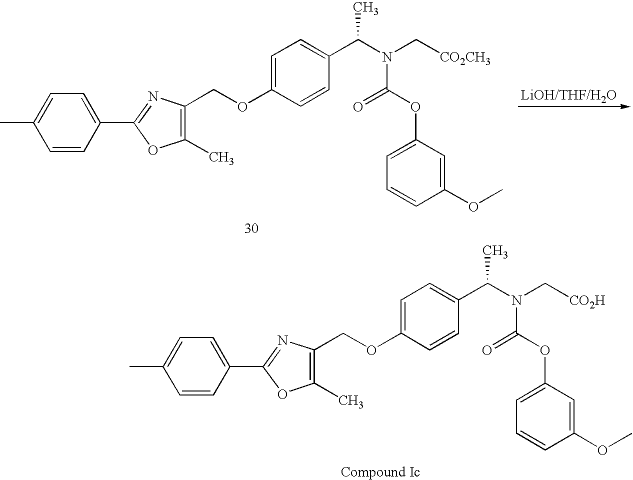

To a solution of carbamate ester 30 (90 g, 165 mmol) in THF (200 mL) was added a solution of LiOH.H₂O (13.9 g, 330 mmol) in H₂O (200 mL). The mixture was warmed to 40° C. and stirred at 40° C. for 3 h, then was cooled to 0° C. The pH of the mixture was adjusted to 1 by addition of concentrated HCl (˜30 mL) at 0° C. The mixture was diluted with EtOAc (500 mL), and the organic phase was washed with H₂O (8×200 mL) and concentrated to half the original volume, during which a white solid formed. This solid was dried in vacuo to give crude carbamate-acid Ic (62 g, 117 mmol) with purity >99%. The mother liquor was collected and concentrated to half the original volume, then was left at RT overnight, after which a white solid was formed. This material was collected, dried under vacuum, and combined with the previous 62 g batch. The combined batches of crude carbamate-acid Ic (75.5 g, 86.3%) had a purity of >99%.

Recrystallization:

Crude carbamate-acid Ic (168.4 g, multiple lots) was dissolved in hot EtOAc (2 L) at reflux, then was concentrated to ˜75% of the original volume. This EtOAc solution was filtered, then was stirred at 60° C. for a further 7 h, cooled to RT and kept at RT for 2 days. A white solid formed, was collected and the solid was washed with EtOAc/hexanes (1:1, 400 mL), followed by hexanes (3×300 mL). The white solid was dried in vacuo at 60° C. for 24 h to give carbamate-acid Ic (157 g, 93% recovered yield) with >99.5% purity and 99.6% ee.

1H NMR (500 MHz, DMSO-d6; 24° C.) δ 12.70 (br. s., 1 H), 7.83 (d, J=8.25 Hz, 2 H), 7.22-7.42 (m, 5 H), 6.96-7.10 (m, 2 H), 6.80 (d, J=7.15 Hz, 1 H), 6.59-6.71 (m, 2 H), 5.31-5.46 (m, 1 H), 4.98 (s, 2 H), 3.72-4.00 (m, 5 H), 2.43 (s, 3 H), 2.36 (s, 3 H), 1.44-1.62 (m, 3 H)

¹³C NMR (126 MHz, DMSO-d6; 24° C.) δ 170.5, 159.9, 159.0, 157.5, 153.9, 153.6, 152.2, 147.1, 140.2, 133.3, 132.7, 131.8, 129.7, 128.4, 128.2, 125.6, 124.2, 114.6, 113.9, 113.7, 111.0, 110.9, 107.7, 107.5, 61.4, 55.3, 54.4, 53.5, 45.3, 44.5, 21.0, 17.8, 16.7, 10.0 (note: extra peaks are due to rotamers as the spectrum was run at 24° C.)

HRMS (M+H)⁺=531.2122 (Δ=−1.8 ppm)

[α]_{D}=−90.07° (c=7.366 mg/mL, CH₃OH, temperature=25° C., λ=589 nm)

Theoretical for (C₃₀H₃₀N₂O₇): C, 67.91%; H, 5.69%; N, 5.28%.

Average found from two tests (C₃₀H₃₀N₂O₇): C, 67.92%; H, 5.76%; N, 5.22%.

Biological Data

In vitro PPAR agonist functional assays were performed by transiently transfecting GAL4-hPPARα-LBD or GAL4-hPPARγ-LBD constructs respectively into HEK293 (human embryonic kidney) cells stably expressing 5×GAL4RE-Luciferase. Data were normalized for efficacy at 1 μM to known agonists (BRL-49653 for hPPARγ and GW-233 1 for hPPARα). Agonist binding results in an increase in luciferase enzyme activity which can be monitored by measuring luminescence upon cell lysing and the addition of luciferin substrate. EC₅₀ values (μM) for PPARα or γ agonist activity were calculated as the concentration of the test ligand (μM) required for the half-maximal fold induction of HEK293 cells. The “intrinsic activity” of a test ligand is defined as its activity at 1 μM (expressed as a percentage) relative to the activity of the primary standards (GW2331 for PPARα and BRL-49653/rosiglitazone for PPARγ respectively, both tested at 1 μM). Compounds of formula I are functional agonists with activities in the range of EC₅₀=1-10 nM against the human PPARγ receptor and EC₅₀=1-10 nM against the human PPARα receptor. The ratios of the PPARα:PPARγ EC₅₀ values of compounds of formula I are between 1:2 and 2:1 in this functional assay. In vitro functional data for Compounds Ia-Ic are shown in the table below.

TABLE 1

| | PPARα EC₅₀ (nM) | PPARγ EC₅₀ (nM) |
|---|---|---|
| Compound | (% efficacy) | (% efficacy) |
| 1a | 6 ± 1 | 3 ± 1 |
| | (92 ± 16%) | (128 ± 19%) |
| 1b | 8 ± 2 | 4 ± 2 |
| | (95 ± 9%) | (126 ± 23%) |
| 1c | 6 ± 2 | 5 ± 2 |
| | (82 ± 10%) | (124 ± 19%) |

It is typically known in the art that PPARγ agonists cause edema both in animals and in the clinic. The present PPAR α/γ dual agonists/activators, which have equivalent human PPARα vs. human PPARγ functional activity in a Gal4 transactivation assay in a HEK (human embryonic kidney) cell line, may be advantageous over other PPARα/γ dual agonists with increased potency at PPARγ than at PPARα (i.e., EC₅₀ PPARγ<<EC₅₀ PPARα) in that the anti-dyslipidemic effects (from activation of PPARα) may be manifested at a sufficiently low dose before the edemagenic effects from activation of PPARγ become unmanageable.

8 week old female db/db mice were dosed orally once daily for 14 days at 10 mg/kg with the Example 1a-1c compounds using a vehicle comprised of 5% 1-methyl-pyrrolidinone, 20% polyethylene glycol (PEG400) and 75% 20 mM dibasic sodium phosphate. Plasma samples were obtained from mice fasted overnight (18 hours after last administration of compound) on day 15. The plasma glucose and triglycerides levels were determined and the percentage reductions in both parameters of drug-treated animal relative to vehicle-treated animals are shown in the table below.

TABLE 2

| Compound (10 | % Reduction in | % Reduction in |
|---|---|---|
| mg/kg orally | Plasma Glucose | Plasma Triglycer- |
| dosed once daily | vs Vehicle- | ides vs Vehicle- |
| for 14 days) | control group | control group |
| 1a | −45% | −45% |
| 1b | −34% | −50% |
| 1c | −34% | −61% |

It is known by one skilled in the art that the compounds of the present invention normalize plasma glucose levels and decrease plasma triglycerides at doses ≧10 mg/kg in rodent models of type 2 diabetes (e.g. the db/db mouse). The typical administration of said compounds is expected to be between 0.1 to 2,000 mg/day in the clinical setting, and is preferably between 0.5 to 100 mg/day. (Reference for db/db mouse as an in vivo rodent model for antidiabetic efficacy: T. Harrity et al, Diabetes, 2006, 55, 240-248).

What is claimed is:

1. A compound having the structure of Formula (I): 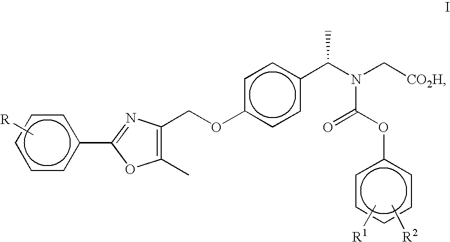
wherein R is hydrogen or C₁-C₄ alkyl; and each of R¹ and R² is independently hydrogen, C₁-C₄ alkyl, halo or C₁-C₄ alkoxy, and salts thereof.

2. A compound having the structure of Formula (Ia):

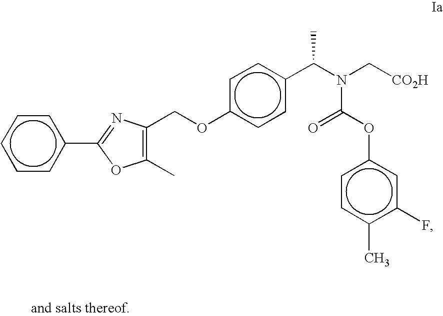

3. A compound having the structure of Formula (Ib):

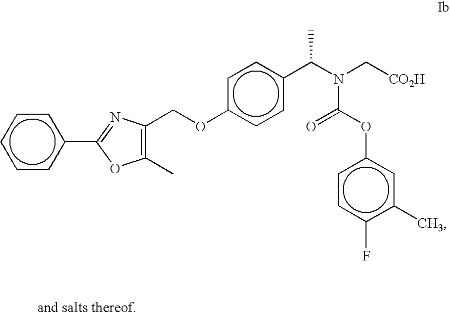

4. A compound having the structure of Formula (Ic):

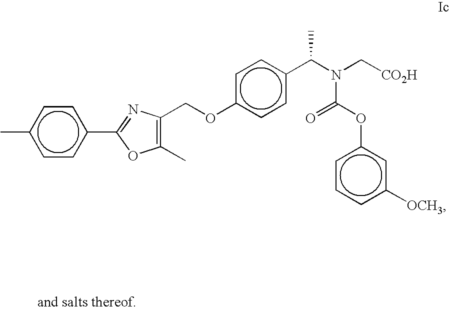

5. A pharmaceutical composition comprising a compound as defined in claim 1 and a pharmaceutically acceptable carrier therefor.

6. A method for treating atherosclerosis which comprises administering to a patient in need of treatment a therapeutically effective amount of a compound as defined in claim 1.

7. A method for lowering blood glucose levels which comprises administering to a patient in need of treatment a therapeutically effective amount of a compound as defined in claim 1.

8. A method for treating diabetes which comprises administering to a patient in need of treatment a therapeutically effective amount of a compound as defined in claim 1.

9. A method for treating dyslipidemia which comprises administering to a patient in need of treatment a therapeutically effective amount of a compound as defined in claim 1.

10. A pharmaceutical combination comprising a compound as defined in claim 9 and a anti-dyslipidemic agent, a lipid modulating agent, an anti-diabetic agent, an anti-obesity agent, an anti-hypertensive agent, a platelet aggregation inhibitor or an anti-osteoporosis agent, or a combination thereof.

11. The pharmaceutical combination as defined in claim 10, comprising said compound and an anti-diabetic agent.

12. The pharmaceutical combination as defined in claim 11, wherein the anti-diabetic agent is 1, 2, 3 or more of a biguanide, a sulfonyl urea, a glucosidase inhibitor, a PPAR γ agonist, a PPAR α/γ dual agonist, an SGLT2 inhibitor, a DP4 inhibitor, an insulin sensitizer, a glucagon-like peptide-1 (GLP-1) or one of its analogs, a cannabinoid receptor 1 (CB-1) antagonist, insulin or a meglitinide, or a combination thereof.

13. The pharmaceutical combination as defined in claim 12, wherein the anti-diabetic agent is 1, 2, 3 or more of metformin, glyburide, glimepiride, glipyride, glipizide, chlorpropamide, gliclazide, acarbose, miglitol, pioglitazone, rosiglitazone, insulin, exenatide, sitagliptin, saxagliptin, vildagliptin, alogliptin, NN-2344, L895645, YM-440, R-119702, AJ9677, repaglinide, nateglinide, KAD1129, AC2993, LY315902, P32/98 or NVP-DPP-728A, or a combination thereof.

14. The pharmaceutical combination as defined in claim 11, wherein said compound is present in a weight ratio to the anti-diabetic agent within the range from about 0.001 to about 100:1.

15. The pharmaceutical combination as defined in claim 14, wherein the anti-obesity agent is a beta 3 adrenergic agonist, a lipase inhibitor, a serotonin (and dopamine) reuptake inhibitor, a thyroid receptor agonist, a cannabinoid receptor 1 (CB-1) antagonist, and aP2 inhibitor or an anorectic agent or a combination thereof.

16. The pharmaceutical combination as defined in claim 15, wherein the anti-obesity agent is orlistat, ATL-962, AJ9677, L750355, CP331648, sibutramine, topiramate, axokine, dexamphetamine, phentermine, phenylpropanolamine, rimonabant, SLV-319, or mazindol or a combination thereof.

17. A method for treating insulin resistance, hyperglycemia, hyperinsulinemia, elevated blood levels of free fatty acids or glycerol, dyslipidemia, obesity, Syndrome X, dysmetabolic syndrome, inflammation, diabetic complications, impaired glucose homeostasis, impaired glucose tolerance, hypertriglyceridemia or atherosclerosis, which comprises administering to a mammalian species in need of treatment thereof a therapeutically effective amount of a pharmaceutical combination as defined in claim 10.

18. A method for treating irritable bowel syndrome, Crohn's disease, gastric ulceritis, osteroporosis, or psoriasis, which comprises administering to a mammalian species in need of treatment thereof a therapeutically effective amount of a compound as defined in claim 1.

19. A compound having the structure of Formula (II), and salts thereof: 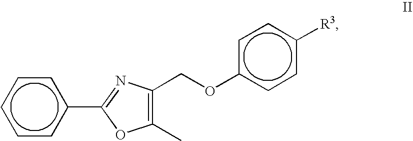
wherein R³ is selected from the group consisting of —COH, —CH═N—(R)—S(O)C(CH₃)₃, —(S)—CH(CH₃)(.H₂O)NH—(R)—S(O)C(CH₃)₃, —(S)—CH(CH₃)NH—(R)—S(O)C(CH₃)₃, —(S)—CH(CH₃)NH₂.HCl, —(S)—CH(CH₃)NH₂, —(S)—CH(CH₃)NH—CH₂CO₂Et, and —(S)—CH(CH₃)NH(.HCl)—CH₂CO₂Et.

20. A compound having the structure of Formula (IV): [chemical structure]
